(19) United States
(12) Patent Application Publication
(10) Pub. No.: US 2002/0128438 A1
(43) Pub. Date: Sep. 12, 2002

(54) DNA cassette for the production of secretable recombinant trimeric TRAIL proteins, tetracycline/ doxycycline-inducible adeno-associated virus vector, their combination and use in gene therapy

(76) Inventors: Dai-Wu Seol, Geoje-si (KR); Timothy R. Billiar, Nevillewood, PA (US)

Correspondence Address: KLAUBER & JACKSON, 411 HACKENSACK AVENUE, HACKENSACK, NJ, 07601

(21) Appl. No.: 09/900,530

(22) Filed: Jul. 6, 2001

(30) Foreign Application Priority Data
Jul. 6, 2000 (KR) 2000-38441

(51) Int. Cl. C07K 14/705; C07H 21/04; C12P 21/02; C12N 5/06

(52) U.S. Cl. 530/350; 435/69.1; 435/320.1; 435/325; 536/23.5

(57) ABSTRACT

The present invention relates to the construction of a TRAIL DNA cassette for the production of a secretable trimeric rTRAIL, the development of pCMVdw vectors and pAAVdw vectors harboring a feed-forward amplification loop type Tet-On system that can be packaged into AAV particles, the preparation of a recombinant vectors by the combination of the TRAIL DNA cassette and the two vectors, and the treatment of diseases including cancer using such vectors.

The present invention provides a TRAIL DNA cassette comprising a secretion signal (SS) sequence, a trimer-forming domain (TFD) and a TRAIL(114-281) coding cDNA.

The TRAIL cassette thus constructed can be cloned into an appropriate expression vector, and subsequently used in the mass production of a secretable recombinant trimeric TRAIL protein or administered to a patient for a gene therapy.

### FIELD OF THE INVENTION

The present invention relates to the construction of a DNA cassette for the production of secretable recombinant proteins, the development of Tetracycline/Doxycycline-inducible Adeno-associated virus vectors, recombinant vectors obtained by the combination of the DNA cassette and the vector, and a gene therapy using these vectors.

Specifically, the present invention relates to the construction of a TRAIL DNA cassette for the production of a secretable trimeric rTRAIL, the development of pCMVdw vectors and pAAVdw vectors harboring a feed-forward amplification loop type Tet-On system that can be packaged into AAV particles, the preparation of recombinant vectors by the combination of the TRAIL DNA cassette and the two vectors, and the treatment of diseases including cancer using such vectors.

### BACKGROUND OF THE INVENTION

Apoptosis, also known as programmed cell death, is an evolutionarily conserved and genetically regulated biological process, and plays an important role in the development and homeostasis of multicellular organisms (Ashkenazi, A. & Dixit, V. M., Death receptors: signaling and modulation, Science 281, 1305-1308 (1998); Nagata, S., Apoptosis by death factor, Cell 88, 355-365 (1997); Salvesen, G. S. & Dixit, V. M., Caspases: intracellular signaling by proteolysis. Cell 91, 443-446 (1997)).

Apoptosis occurs under both physiologic and pathophysiologic circumstances. When apoptosis is dysregulated, diseases often ensue (Thompson, C. B., Apoptosis in the pathogenesis and treatment of disease, Science 267, 1456-1462 (1995); Rimon, G., Bazenet, C. E., Philpott, K. L. & Rubin, L. L., Increased surface phosphatidylserine is an early marker of neuronal apoptosis., J. Neurosci. Res. 48, 563-570 (1997); Krams, S. M. & Martinez, O. M., Apoptosis as a mechanism of tissue injury in liver allograft rejection. Semin. Liver Dis. 18, 153-167 (1998); Olivetti, G. et al., Apoptosis in the failing human heart, N. Engl. J. Med. 336, 1131-1141 (1997); Darzykiewicz, Z., Apoptosis in antitumor strategies: Modulation of cell cycle or differentiation, J. Cell. Biol. 58, 151-159 (1995)). Diseases associated with excessive apoptosis include AIDS, neurodegenerative disorders (e.g., Alzheimer's disease), ischemia/reperfusion injury, progression of heart failure in cardiomyopathies, viral infections (e.g., chronic hepatitis), toxin-induced liver disease and organ transplantation rejections.

Diseases associated with too little apoptosis include cancer and autoimmune disorders.

Among the many factors known to induce apoptosis, TNF-α, FasL (Fas ligand) and TRAIL (Tumor necrosis factor-related apoptosis-inducing ligand) are included as factors that are produced in the organisms and induce apoptosis as a physiological process. Recently, there have been many studies for the treatment of diseases that have been considered hard to cure, such as cancer, using the apoptosis mechanism.

In the apoptotic signaling pathway induced by TNF-α, the activation of TNF-R1, a TNF-α receptor, by the binding of TNF-α, elicits the recruitment of a cellular adaptor protein TRADD (Hsu, H., Xiong, J. & Goeddel, D. V., The TNF receptor 1-associated protein TRADD signals cell death and NF-kappa B activation, Cell 81, 495-504 (1995)) to the activated TNF-R1. TRADD recruits other cellular proteins such as FADD, TNF receptor-associate factor-2 (TRAF-2) (Hsu, H., Shu, H. B., Pan, M. G. & Goeddel, D. V., TRADD-TRAF2 and TRADD-FADD interactions define two distinct TNF receptor 1 signal transduction pathways. Cell 84, 299-308 (1996)) and receptor interacting protein (RIP) (Hsu, H., Huang, J., Shu, H. B., Baichwal, V. & Goeddel, D. V., TNF-dependent recruitment of the protein kinase RIP to the TNF receptor-1 signaling complex. Immunity 4, 387-396 (1996)). FADD directly interacts with procaspase-8 leading to its proteolytic activation (Boldin, M. P., Goncharov, T. M., Goltsev, Y. V. & Wallach, D., Involvement of MACH, a novel MORT1/FADD-interacting protease, in Fas/APO-1- and TNF receptor-induced cell death. Cell 85, 803-815 (1996); Muzio, M. et al., FLICE, a novel FADD-homologous ICE/CED-3-like protease, is recruited to the CD95 (Fas/APO-1) death-inducing signaling complex. Cell 85, 817-827 (1996); Srinivasula, S. M., Ahmad, M., Fernandes-Alnemri, T., Litwack, G. & Alnemri, E. S., Molecular ordering of the Fas-apoptotic pathway: the Fas/APO-1 protease Mch5 is a CrmA-inhibitable protease that activates multiple Ced-3/ICE-like cysteine proteases. Proc. Natl. Acad. Sci. USA 93, 14486-14491 (1996)). TRAF-2 and RIP stimulate the signal pathways leading to activation of NF-κB and Jun kinase, which have been shown to inhibit apoptosis in some cell types (Liu, Z. G., Hsu, H., Goeddel, D. V. & Karin, M. Dissection of TNF receptor 1 effector functions: JNK activation is not linked to apoptosis while NF-kappaB activation prevents cell death. Cell 87, 565-576 (1996). Kelliher, M. A. et al., The death domain kinase RIP mediates the TNF-induced NF-kappaB signal. Immunity 8, 297-303 (1998)). Activation of NF-κB also leads to the induction of genes involved in proinflammatory and immune reactions (Lenardo, M. J., Fan, C. M., Maniatis, T. & Baltimore, D., The involvement of NF-kappa B in beta-interferon gene regulation reveals its role as widely inducible mediator of signal transduction. Cell 57, 287-294 (1989); Libermann, T. A. & Baltimore, D., Activation of interleukin-6 gene expression through the NF-kappa B transcription factor. Mol. Cell. Biol. 10, 2327-2334 (1990); Sha, W. C., Liou, H. C., Tuomanen, E. I. & Baltimore, D., Targeted disruption of the p50 subunit of NF-kappaB leads to multifocal defects in immune responses. Cell 80, 321-330 (1995); Kontgen, F. et al., Mice lacking the c-rel proto-oncogene exhibit defects in lymphocyte proliferation, humoral immunity, and interleukin-2 expression. Genes Dev. 9, 1965-1977 (1995)). These observations indicate that TNF-α activates both apoptotic and anti-apoptotic signal pathways. Thus, TNF-α induces limited apoptosis in a number of cell systems unless the anti-apoptotic signal pathway is blocked (Beg, A. A. & Baltimore, D., An essential role for NF-kappaB in preventing TNF-alpha-induced cell death. Science 274, 782-784 (1996); Wang, C. Y., Mayo, M. W. & Baldwin, A. S. J., TNF- and cancer therapy-induced apoptosis: potentiation by inhibition of NF-kappaB. Science 274, 784-787 (1996); Van Antwerp, D. J., Martin, S. J., Kafri, T., Green, D. R. & Verma, I. M., Suppression of TNF-alpha-induced apoptosis by NF-kappaB. Science 274, 787-789 (1996)).

Unlike TNF-R1, the activated Fas recruits only FADD as an adaptor molecule (Boldin, M. P. et al., A novel protein that interacts with the death domain of Fas/APO1 contains a sequence motif related to the death domain. J. Biol. Chem. 270, 7795-7798 (1995); Chinnaiyan, A. M., O'Rourke, K., Tewari, M. & Dixit, V. M., FADD, a novel death domain-containing protein, interacts with the death domain of Fas and initiates apoptosis. Cell 81, 505-512 (1995); Chinnaiyan, A. M. et al., FADD/MORT1 is a common mediator of CD95 (Fas/APO-1) and tumor necrosis factor receptor-induced apoptosis. J. Biol. Chem. 271, 4961-4965 (1996); Juo, P., Kuo, C. J., Yuan, J. & Blenis, J., Essential requirement for caspase-8/FLICE in the initiation of the Fas-induced apoptotic cascade. Curr. Biol. 8, 1001-1008 (1998)). As a result, Fas activates only the apoptotic signaling pathways. This probably explains the observation that FasL has a stronger apoptotic activity than TNT-α. Once activated, caspase-8 initiates the caspase cascade leading to cleavage of cytosolic, cytoskeletal as well as nuclear proteins and DNA. Studies have demonstrated that the Bcl-2 family member Bid is cleaved by caspase-8. The cleaved Bid was shown to induce cytochrome c release from mitochondria (Luo, X., Budihardjo, I., Zou, H., Slaughter, C. & Wang, X., Bid, a Bcl2 interacting protein, mediates cytochrome c release from mitochondria in response to activation of cell surface death receptors. Cell 94, 481-490 (1998); Li, H., Zhu, H., Xu, C. J. & Yuan, J., Cleavage of BID by caspase 8 mediates the mitochondrial damage in the Fas pathway of apoptosis. Cell 94, 491-501 (1998)). Once released, cytochrome c binds to Apaf-1 and participates in caspase-9 activation (Li, P. et al., Cytochrome c and dATP-dependent formation of Apaf-1/caspase-9 complex initiates an apoptotic protease cascade. Cell 91, 479-489 (1997); Pan, G., O'Rourke, K. & Dixit, V. M., Caspase-9, Bcl-XL, and Apaf-1 form a ternary complex. J. Biol. Chem. 273, 5841-5845 (1998)). The activated caspase-9 is then able to activate caspase-3 (Li, P. et al., Cytochrome c and dATP-dependent formation of Apaf-1/caspase-9 complex initiates an apoptotic protease cascade. Cell 91, 479-489 (1997)), which in turn liberates a DNase termed CAD (caspase-activated DNase) from an inhibitor of CAD (ICAD/DFF-45) by cleaving the ICAD protein (Enari, M. et al., A caspase-activated DNase that degrades DNA during apoptosis, and its inhibitor ICAD. Nature 391, 43-50 (1998); Sakahira, H., Enari, M. & Nagata, S., Cleavage of CAD inhibitor in CAD activation and DNA degradation during apoptosis. Nature 391, 96-99 (1998); Liu, X. et al., The 40-kDa subunit of DNA fragmentation factor induces DNA fragmentation and chromatin condensation during apoptosis. Proc. Natl. Acad. Sci. USA 95, 8461-8466 (1998)). This process leads to DNA degradation, a hallmark event in apoptosis.

TRAIL (tumor necrosis factor-related apoptosis-inducing ligand), another apoptosis inducing factor, is a member of the TNF family. As other TNF family members such as TNF-α and Fas ligand (FasL), TRAIL is a type II transmembrane ligand molecule (Wiley, S.R. et al., Identification and Characterization of a New Member of the TNF Family that Induces Apoptosis. Immunity 3, 673-682 (1995); Pitti, R. M. et al., Induction of apoptosis by Apo-2 ligand, a new member of the tumor necrosis factor cytokine family. J. Biol. Chem. 271, 12687-12690 (1996)). Recent studies (Cha, S. S. et al., 2.8 A resolution crystal structure of human TRAIL, a cytokine with selective antitumor activity. Immunity 11, 253-261 (1999); Hymowitz, S. G. et al., Triggering cell death: the crystal structure of Apo2L/TRAIL in a complex with death receptor 5. Mol. Cell 4, 563-571 (1999)) about the crystal structure of the TRAIL protein revealed that soluble TRAIL protein truncated in its transmembrane domain forms a homotrimer. Trimerization was shown to be a required process for a recombinant TRAIL (rTRAIL) to obtain apoptotic capacity (Hymowitz, S. G. et al., A unique zinc-binding site revealed by a high-resolution X-ray structure of homotrimeric Apo2L/TRAIL. Biochemistry 39, 633-640 (2000)).

Despite the physiological and therapeutic importance of TRAIL, relatively little is known about the death signaling events for TRAIL. Recent data suggest an essential role of caspase-8 in TRAIL-induced apoptosis, pointing to the similarity of TRAIL-R-mediated signaling with Fas-mediated signaling (Seol, D. W., Li, J., Seol, M. H., Talanian, R. V. & Billiar, T. R., Signaling events triggered by tumor necrosis factor-related apoptosis-inducing ligand (TRAIL): caspase-8 is required for TRAIL-induced apoptosis. Manuscript submitted).

Four different TRAIL receptors have been thus far identified, including DR4/TRAIL-R1 (Pan, G. et al., The receptor for the cytotoxic ligand TRAIL. Science 276, 111-113 (1997)), DR5/TRAIL-R2 (Pan, G. et al., An antagonist decoy receptor and a death domain-containing receptor for TRAIL. Science 277, 815-818 (1997); Sheridan, J. P. et al., Control of TRAIL-induced apoptosis by a family of signaling and decoy receptors. Science 277, 818-821 (1997); MacFarlane, M. et al., Identification and molecular cloning of two novel receptors for the cytotoxic ligand TRAIL. J. Biol. Chem. 272, 25417-25420 (1997)) and two decoy receptors (DcR1 and DcR2) (Pan, G. et al., An antagonist decoy receptor and a death domain-containing receptor for TRAIL. Science 277, 815-818 (1997); Sheridan, J. P. et al., Control of TRAIL-induced apoptosis by a family of signaling and decoy receptors. Science 277, 818-821 (1997); MacFarlane, M. et al., Identification and molecular cloning of two novel receptors for the cytotoxic ligand TRAIL. J. Biol. Chem. 272, 25417-25420 (1997); Marsters, S. A. et al., A novel receptor for Apo2L/TRAIL contains a truncated death domain. Curr. Biol. 7, 1003-1006 (1997)). DR4 and DR5 are intact functional TRAIL receptors through which the apoptosis-inducing activity of TRAIL is transmitted into the cytoplasm. DcR1 and DcR2 are truncated TRAIL receptors where the cytoplasmic regions containing the death domains are deleted. Thus, over-expression of DcR1 and DcR2 blocks the function of DR4 and DR5, probably by competing with DR4 or DR5 for TRAIL (Pan, G. et al., An antagonist decoy receptor and a death domain-containing receptor for TRAIL. Science 277, 815-818 (1997); Sheridan, J. P. et al., Control of TRAIL-induced apoptosis by a family of signaling and decoy receptors. Science 277, 818-821 (1997); MacFarlane, M. et al., Identification and molecular cloning of two novel receptors for the cytotoxic ligand TRAIL. J. Biol. Chem. 272, 25417-25420 (1997); Marsters, S. A. et al., A novel receptor for Apo2L/TRAIL contains a truncated death domain. Curr. Biol. 7, 1003-1006 (1997)).

Although TRAIL is a TNF family member, it has some notable difference with TNF-α and FasL. Unlike Fas whose expression is limited to certain tissues, TRAIL receptors are widely expressed (Pan, G. et al., The receptor for the cytotoxic ligand TRAIL. Science 276, 111-113 (1997); Pan, G. et al., An antagonist decoy receptor and a death domain-containing receptor for TRAIL. Science 277, 815-818 (1997); Sheridan, J. P. et al., Control of TRAIL-induced apoptosis by a family of signaling and decoy receptors. Science 277, 818-821 (1997); MacFarlane, M. et al., Identification and molecular cloning of two novel receptors for the cytotoxic ligand TRAIL. J. Biol. Chem. 272, 25417-25420 (1997)), thus most tissues and cell types may be TRAIL targets. TRAIL has a unique selectivity for triggering apoptosis in tumor cells but not in normal cells in vivo. Therefore, in contrast to FasL or an agonistic Fas antibody that induces fulminant massive liver damage in systemic delivery (Ogasawara, J. et al., Lethal effect of the anti-Fas antibody in mice. Nature 364, 806-809 (1993); Galle, P. R. et al., Involvement of the CD95 (APO-1/Fas) receptor and ligand in liver damage. J. Exp. Med. 182, 1223-1230 (1995)), TRAIL exhibited no detectable cytotoxicity in mice (Walczak, H. et al., Tumoricidal activity of tumor necrosis factor-related apoptosis-inducing ligand in vivo. Nat. Med. 5, 157-163 (1999)) and monkey (Ashkenazi, A. et al., Safety and antitumor activity of recombinant soluble Apo2 ligand. J. Clin. Invest. 104, 155-162 (1999)). HIV-1-infected T cells were also shown to be more susceptible than uninfected T cells to TRAIL (Jeremias, I., Herr, I., Boehler, T. & Debatin, K. M., TRAIUApo-2-ligand-induced apoptosis in human T cells. Eur. J. Immunol. 28, 143-152 (1998)). In addition, TRAIL-induced apoptosis does not depend upon p53 status that is considered a critical factor in cancer therapies using chemotherapeutic agents or radiation (Seol, D. W., Seol, M. H. & Billiar, T. R., Tumor necrosis factor-related apoptosis-inducing ligand (TRAIL) induces apoptosis independent of p53 status. Manuscript in preparation). These features indicate that rTRAIL protein is a very promising therapeutic treatment of cancers and AIDS.

However, there are technical problems to be solved before using rTRAIL as an anticancer drug. First, the production of a sufficient amount of high-quality rTRAIL is difficult. Even the production yield from bacterial cells is insufficient. Second, even if a sufficient amount of rTRAIL can be purified from bacterial cells via improved purification methods, the elimination of contaminants including endotoxin presents further problems. Third, it has not yet been proved that bacteria-produced rTRAIL is identical to the mammalian cell-produced protein in all biological aspects. Therefore, a safer and more reliable way to produce rTRAIL could be possible by using transfected mammalian cells. However, if the rTRAIL protein produced in this way is used as therapeutic, a high cost burden would be imparted upon patients due to the expensive nature of this approach. In addition, protein therapy using rTRAIL, like all currently generalized anticancer drug therapies, would be performed in a manner by which patients come to hospital periodically to continue the administration of the therapeutic until the treatment is terminated. This too results in a high cost burden.

Adeno-associated virus (AAV) is a parvovirus that is naturally defective, non-enveloped and non-pathogenic (Berns, K. I. & Adler, S., Separation of two types of adeno-associated virus particles containing complementary polynucleotide chains. J. Virol. 9, 394-396 (1972); Berns, K. I. & Rose, J. A., Evidence for a single-stranded adenovirus-associated virus genome: isolation and separation of complementary single strands. J. Virol. 5, 693-699 (1970)). The AAV replication cycle is composed of two distinct phases, a latent phase and a productive phase. In the presence of a helper virus such as adenovirus (Berns, K., Parvoviridae and their replication, in: Fields, B. N., Knipe, D. M. (Eds), Virology, 2nd edition. Vol. 2. Raven Press, New York, pp. 1743-1764 (1990); Muzyczka, N., Use of adeno-associated virus as a general transduction vector. Curr. Topics Microbiol. Immunol. 158, 97-129 (1992); Berns, K.I. & Giraud, C., Biology of the adeno-associated virus, in: Berns, K. I., Giraud, C. (Eds.), Adeno-associated virus (AAV) vectors in gene therapy. Vol. 218. Springer, Berlin, pp. 1-24. (1996)), AAV replicates generating progeny virions. In the absence of a helper virus, AAV integrates its genome into a specific site on chromosome 19 causing no deleterious effects and remains integrated until a subsequent helper virus infection rescues the virus from the latent state (Kotin, R. M. et al., Site-specific integration by adeno-associated virus. Proc. Natl. Acad. Sci. USA 87, 2211-2215 (1990)).

The AAV genome is a linear, single-stranded DNA, which contains a 145-base terminal repeat (TR) sequence at each end (Srivastava, A., Lusby, E. W. & Berns, K. I., Nucleotide sequence and organization of the adeno-associated virus 2 genome. J. Virol. 45, 555-564 (1983)).

The TR sequence serves as the origin of DNA replication and the only known cis-acting element required for packaging recombinant AAV (rAAV) genomes into infectious virions. The TR elements flank two open reading frames (ORFs). The left ORF (or rep gene) encodes four non-structural proteins called Rep78, 68, 52 and 40 that are essential for viral DNA replication and assembly. The right ORF (or cap gene) encodes three structural proteins VP1, VP2 and VP3 (Bems, K., Parvoviridae and their replication, in: Fields, B. N., Knipe, D. M. (Eds), Virology, 2nd edition. Vol. 2. Raven Press, New York, pp. 1743-1764. (1990); Muzyczka, N., Use of adeno-associated virus as a general transduction vector. Curr. Topics Microbiol. Immunol. 158, 97-129 (1992); Berns, K. I. & Giraud, C., Biology of the adeno-associated virus, in: Berns, K. I., Giraud, C. (Eds.), Adeno-associated virus (AAV) vectors in gene therapy. Vol. 218. Springer, Berlin, pp. 1-24. (1996)).

rAAV vector construction typically involves removing the rep and cap genes and inserting the transgene of interest between the TR elements. The resulting vector plasmid is then cotransfected into tissue culture cells along with a packaging plasmid that expresses the AAV rep and cap genes (Xiao, X., Li, J. & Samulski, R. J., Production of high-titer recombinant adeno-associated virus vectors in the absence of helper adenovirus. J. Virol. 72, 2224-2232 (1998)). Helper functions for efficient AAV replication can be provided by adenovirus (Samulski, R. J., Chang, L. S. & Shenk, T. Helper-free stocks of recombinant adeno-associated viruses: normal integration does not require viral gene expression. J. Virol. 63, 3822-3828 (1989)) or a helper plasmid expressing helper genes of adenovirus (Xiao, X., Li, J. & Samulski, R. J., Production of high-titer recombinant adeno-associated virus vectors in the absence of helper adenovirus. J. Virol. 72, 2224-2232 (1998); Grimm, D., Kern, A., Rittner, K. & Kleinschmidt, J. A. Novel tools for production and purification of recombinant adenoassociated virus vectors. Hum. Gene Ther. 9, 2745-2760 (1998)). With the aid of these helper genes, AAV particles packaged from recombinant DNAs can be produced in a large scale from cultured cells.

AAV's ability to be integrated site-specifically, its natural defectiveness and its lack of pathogenicity have led to its potential use as a gene therapy vector. AAV's capacity to integrate genome also makes possible a long-term target gene expression. rAAV. vectors can transduce cells in culture as well as a variety of different tissues in vivo, such as lung (Flotte, T. R. et al., Stable in vivo expression of the cystic fibrosis transmembrane conductance regulator with an adeno-associated virus vector. Proc. Natl. Acad. Sci. USA 90, 10613-10617 (1993); Conrad, C. K. et al., Safety of single-dose administration of an adeno-associated virus (AAV)-CFTR vector in the primate lung. Gene Ther. 3, 658-668 (1996); Halbert, C. L. et al., Transduction by adeno-associated virus vectors in the rabbit airway: efficiency, persistence, and readministration. J. Virol. 71, 5932-5941 (1997)), brain (Kaplitt, M. G. et al., Long-term gene expression and phenotypic correction using adeno-associated virus vectors in the mammalian brain. Nat. Genet. 8, 148-154 (1994)), muscle (Xiao, X., Li, J. & Samulski, R. J., Efficient long-term gene transfer into muscle tissue of immunocompetent mice by adeno-associated virus vector. J. Virol. 70, 8098-8108 (1996); Rivera, V. M. et al., Long-term regulated expression of growth hormone in mice after intramuscular gene transfer. Proc. Natl. Acad. Sci. USA 96, 8657-8662 (1999)), retina (Flannery, J. G. et al., Efficient photoreceptor-targeted gene expression in vivo by recombinant adeno-associated virus. Proc. Natl. Acad. Sci. USA 94, 6916-6921 (1997)), central nervous system (Peel, A. L., Zolotukhin, S., Schrimsher, G. W., Muzyczka, N. & Reier, P. J., Efficient transduction of green fluorescent protein in spinal cord neurons using adeno-associated virus vectors containing cell type-specific promoters. Gene Ther. 4, 16-24 (1997)) and liver (Xiao, W. et al., Adeno-associated virus as a vector for liver-directed gene therapy. J. Virol. 72, 10222-10226 (1998)).

As described above, although TRAIL is attracting attention as a promising anticancer drug, it has various problems in terms of applicability. The present invention has now provided a TRAIL DNA cassette for the production of novel secretable rTRAIL, a constitutive expression vector, an AAV vector system suitable for a gene therapy, and a gene therapy using them.

### SUMMARY OF THE INVENTION

The present invention provides a DNA cassette encoding a recombinant TRAIL (amino acid 114-281) protein that can be secreted into culture media or the circulation system in vivo and has a greatly enhanced homotrimer-forming function.

More specifically, the present invention provides a DNA cassette comprising a secretion signal (SS) sequence for the secretion of recombinant TRAIL protein, a trimerforming domain (TFD) for promoting the trimer formation, and a cDNA encoding TRAIL(114-281).

The present invention also provides a recombinant expression vector comprising the DNA cassette, and a cell line transformed with the recombinant expression vector.

In another aspect, the present invention provides a recombinant TRAIL(114-281) protein recovered from the transformed cell line.

In yet another aspect, the present invention also provides a pCMVdw vector that can effect the constitutive expression of an inserted foreign gene.

More specifically, the present invention provides recombinant vectors pCMVdwSEC2ILZTRAIL(114-281) and pCMVdwSEC(CV)ILZTRAIL(114-281), which can effect the constitutive expression of a secretable trimeric TRAIL protein.

In addition, the present invention provides a pharmaceutical composition comprising pCMVdwSEC2ILZTRAIL(114-281) or pCMVdwSEC(CV)ILZTRAIL(114-281) for the treatment of such diseases as cancer, AIDS and the like.

The present invention further provides an adeno-associated virus (AAV) vector that can effect the inducible expression of an inserted foreign gene.

More specifically, the present invention provides an adeno-associated virus vector system pAAVdw, which can achieve the maximal expression of a foreign gene by a feedforward amplification loop manner in response to the induction by Tetracycline/Doxycycline.

The present invention provides pAAVdwSEC2ILZTRAIL(114-281) and pAAVdwSEC(CV)ILZTRAIL(114-281), recombinant expression vectors which comprise the TRAIL DNA cassette of the present invention and can be utilized in the expression of recombinant trimeric TRAIL in a Tet-On-inducible manner.

The present invention also provides a pharmaceutical composition comprising a pAAVdw vector.

More specifically, the present invention provides a pharmaceutical composition comprising pAAVdwSEC2ILZTRAIL(114-281) or pAAVdwSEC(CV)ILZTRAIL(114-281) vector for the treatment of such diseases as cancer, AIDS and the like.

The present invention further provides virus particles packaged from a pAAVdw vector and a pharmaceutical composition comprising the particles.

More specifically, the present invention provides virus particles packaged from pAAVdwSEC2ILZTRAIL(114-281) or pAAVdwSEC(CV)ILZTRAIL(114-281) vector, and a pharmaceutical composition comprising the particles for the treatment of such diseases as cancer, AIDS and the like.

The present invention provides a pharmaceutical composition that can be utilized in a combination therapy for cancer or AIDS, comprising the recombinant TRAIL protein produced by the DNA cassette of the present invention and Actinomycin D (ActD).

### BRIEF DESCRIPTION OF THE DRAWINGS

FIG. 1a shows the nucleotide sequence and amino acid sequence of SEC2 which is derived from human Fibrilin-1 as a secretion signal for the secretion of a protein produced in cells.

FIG. 1b shows the nucleotide sequence and amino acid sequence of SEC(CV) which is derived from human Growth Hormone as a secretion signal for the secretion of a protein produced in cells, where a Furin-specific cleavage sequence has been added to the C-terminus of the SEC(CV) for the ease of cleavage of the secretion signal.

FIG. 2 shows the sequence of the Furin-specific cleavage site, where the cleavage site is indicated by an arrow.

FIG. 3 shows the nucleotide sequence and amino acid sequence of the trimer-forming domain ILZ.

FIG. 4 shows the nucleotide sequence and amino acid sequence of the human TRAIL cDNA(114-281).

FIG. 5 shows the nucleotide sequence of the multiple cloning site of the pCMVdw vector, a mammalian expression vector inducible by a cytomegalovirus (CMV) promoter.

FIG. 6 shows the nucleotide sequence and amino acid sequence of FLAG-tag.

FIG. 7 shows the nucleotide sequence and amino acid sequence of His-tag.

FIG. 8a shows the nucleotide sequence and amino acid sequence of SEC2ILZ.

FIG. 8b shows the nucleotide sequence and amino acid sequence of HisTRAIL(114-281).

FIG. 8c shows the nucleotide sequence and amino acid sequence of SEC2TRAIL(114-281).

FIG. 8d shows the nucleotide sequence and amino acid sequence of FLAGILZTRAIL(114-281).

FIG. 8e shows the nucleotide sequence and amino acid sequence of SEC2ILZTRAIL(114-281).

FIG. 8f shows the nucleotide sequence and amino acid sequence of SEC(CV)ILZTRAIL(114-281).

FIG. 8g depicts expression vectors obtained from the sequences shown in FIG. 8a to 8f, inserted alone or in combination into a pCMVdw vector, the results from a HeLa cell killing activity test, and a photograph showing the cell killing results.

FIG. 9a depicts a photograph of a standard Western blot assay showing the expression and secretion of a TRAIL protein containing the SEC2 secretion signal sequence.

FIG. 9b depicts a photograph of a standard Western blot assay showing the expression, secretion and cleavage of a TRAIL protein containing the SEC(CV) secretion signal sequence.

FIG. 10a is a schematic representation of the procedures for constructing a Tetracycline/Doxycycline-inducible pAAVdw vector.

FIG. 10b shows the nucleotide sequence of a pTRE vector portion corresponding to the “TetO-mP” of the pAAVdw vector.

FIG. 10c shows the nucleotide sequence of the left-arm “TetO-mP” of the pAAVdw vector.

FIG. 10d shows the nucleotide sequence of the right-arm “TetO-mP” of the pAAVdw vector.

FIG. 10e shows the nucleotide sequence of rtTA of the pAAVdw vector.

FIG. 10f shows the nucleotide sequence of TetO-mP-rtTA of the pAAVdw vector.

FIG. 10g schematically depicts principles of the feed-forward amplification expression of a target gene (gfp) in the pAAVdw vector.

FIG. 10h is a photograph of a Western blot using a GFP-recognizing antibody showing the Tetracycline-inducibility of a target gene (gfp) in the pAAVdw vector.

FIG. 11a is a schematic representation of the procedures for constructing a Tetracycline/Doxycycline-inducible pAAVdw-TRAIL vector for the production of a secretable trimeric TRAIL.

FIG. 11b shows test results for the pAAVdwSEC2IWLZTRAIL(114-281) and pAAVdwSEC(CV)ILZTRAIL(14-281) vectors.

FIG. 12 shows the results of an activity test for AAV particles harboring a DNA cassette for producing a secretable trimeric TRAIL, and a feed-forward amplification loop type Tet-On system.

FIG. 13 shows the test results for target cell killing, indicating that Actinomycin D (ActD) enhances the TRAIL-induced apoptosis.

### DETAILED DESCRIPTION OF THE INVENTION

The present invention relates to the construction of a TRAIL DNA cassette for the production of a secretable trimeric rTRAIL, the development of pCMVdw vectors and pAAVdw vectors harboring a feed-forward amplification loop type Tet-On system that can be packaged into AAV particles, the preparation of recombinant vectors by the combination of the TRAIL DNA cassette and the two vectors, and the treatment of diseases including cancer using such vectors.

In one aspect, the present invention is characterized by a TRAIL DNA cassette comprising a secretion signal (SS) sequence, a trimer-forming domain (TFD) and a TRAIL(114-281) coding cDNA.

A secretion signal sequence is required for a protein produced in a cell to be secreted into the extracellular space. Thus, an appropriate secretion signal (SS) sequence has been identified which can facilitate secretion of the rTRAIL to the circulation (or culture medium in cell culture).

If an rTRAIL protein can be secreted into the extracellular space, the rTRAIL produced intracellularly in vivo can be secreted to the circulation. In this case, it would be possible to directly administer to a patient a recombinant vector containing a TRAIL gene as a therapeutic agent in place of an rTRAIL protein, using a so-called gene therapy. If an rTRAIL protein can be secreted into the extracellular space, the isolation and purification of rTRAIL would become far easier even if the isolated and purified rTRAIL is directly used as a therapeutic agent. That means that rTRAIL can be produced from the culture medium of cells transformed with a recombinant vector, not from the cytoplasm of the cells. Consequently, a highly pure rTRAIL can be produced without the problem of contamination by cell debris because a secretable rTRAIL would not be subject to a cell disruption step for the purpose of isolation and purification.

Accordingly, a secretion signal sequence was incorporated into the cassette so that the rTRAIL produced by the recombinant vector can be secreted directly into the circulation or the culture medium.

The fusion of a secretion signal sequence to an rTRAIL according to the present invention represents one of the essential technical features of the present invention, reaching beyond a simple production of a secretable protein. This is because in contrast to known studies and medicinal development using rTRAIL which utilized the rTRAIL protein by its isolation and purification, the present invention enables, by fusing a secretion signal sequence to a TRAIL sequence, the establishment of a gene therapy using the resulting gene sequence. In other words, the present invention has made a progress of the rTRAIL therapy development from the level of protein-based development to the level of gene therapy-based developments by using a secretion signal sequence.

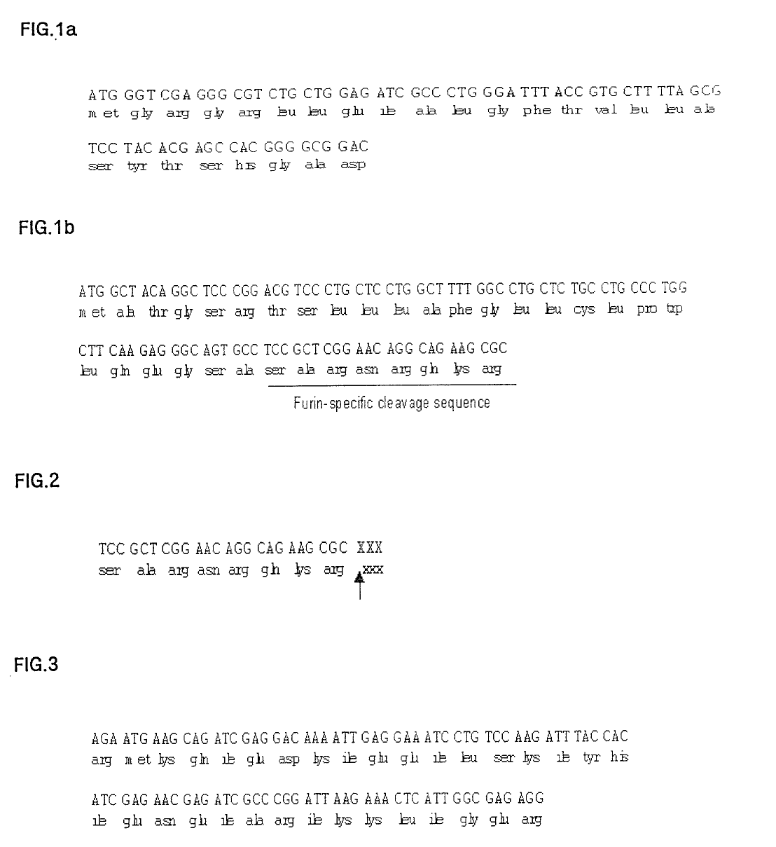

The secretion signal sequences used in the present invention are SEC2 signal sequence (FIG. 1a) and SEC(CV) signal sequence (FIG. 1b). The former was derived from human Fibrilin-1 (Ritty, T. M., Broekelmann, T., Tisdale, C., Milewicz, D. M. & Mecham, R. P., Processing of the fibrillin-1 carboxyl-terminal domain. J. Biol. Chem. 274, 8933-8940 (1999)) and the latter came from human Growth Hormone (Martial, J. A., Hallewell, R. A., Baxter, J. D. & Goodman, H. M., Human growth hormone: complementary DNA cloning and expression in bacteria. Science 205, 602-607 (1979)).

Other secretion signal sequences may also be used and nucleotides including restriction enzyme sites can be added to the 5′ or 3′ terminal of respective secretion signal sequence, to facilitate the incorporation of such sequences into the DNA cassette.

A Furin-specific cleavage sequence (Denault, J. B. & Leduc, R., Furin/PACE/SPC1: a convertase involved in exocytic and endocytic processing of precursor proteins. FEBS Lett. 379, 113-116 (1996)) was added to the carboxyl terminus of the SEC(CV) to facilitate cleavage of the secretion signal sequence and minimize the resulting immune response when used as a therapeutic agent. Furin is one of the best characterized mammalian subtilisin-like proteases. This enzyme is known to cleave a specific site of the recognition sequence. The arrow in FIG. 2 indicates the cleavage site. Other Furin-specific cleavage sequences (Denault, J. B. & Leduc, R., Furin/PACE/SPC1: a convertase involved in exocytic and endocytic processing of precursor proteins. FEBS Lett. 379, 113-116 (1996); Groskreutz, D. J., Sliwkowski, M. X. & Gorman, C. M., Genetically engineered proinsulin constitutively processed and secreted as mature, active insulin. J. Biol. Chem. 269, 6241-5245 (1994)) can be used. Nucleotides including restriction enzyme sites can be added to the 5′ or 3′ terminal of the Furin-specific cleavage sequences. In addition, nucleotides including restriction enzyme sites can also be added between the secretion signal sequence of SEC(CV) and the Furin-specific cleavage sequences.

Other protease-specific cleavage sites can be added to the secretion signal sequences.

In order to incorporate into the cassette an artificial trimer-forming domain that can promote the trimerization of the rTRAIL protein as the second component of the cassette, the SS sequence was fused to an artificial trimer-forming domain (TFD) such as a mutant leucine zipper (LZ). Trimerization is required for an rTRAIL to have an apoptosis-inducing activity. Although soluble rTRAIL tends to form a trimer, trimerization is significantly enhanced when an artificial trimer-forming domain is fused to the soluble rTRAIL (Walczak, H. et al., Tumoricidal activity of tumor necrosis factor-related apoptosis-inducing ligand in vivo. Nat. Med. 5, 157-163 (1999)).

The trimer-forming domain used in the present invention is a variant of a GCN4 leucine zipper, Isoleucine Zipper (ILZ) (Harbury, P. B., Kim, P. S. & Alber, T., Crystal structure of an isoleucine-zipper trimer. Nature 371, 80-83 (1994)), and its amino acid sequence and nucleotide sequence are shown in FIG. 3.

ILZ forms a homotrimer and, when fused to a protein, promotes trimerization of the protein.

A variant of the sequence shown in FIG. 3 is still considered as the ILZ sequence and can be used in the cassette of the present invention, even if the sequence is different from the ILZ sequence shown in FIG. 3 due to the deletion, insertion or substitution of one or more nucleotides, if the translated amino acid sequence retains a sequence homology of at least 80% to the amino acid sequence in FIG. 3 and the activity as a trimer-forming domain.

Nucleotides including restriction enzyme sites can be incorporated to the 5′ and 3′ terminal of the ILZ coding sequence.

Trimer-forming domains other than ILZ, such as trimeric LZs (Walczak, H. et al., Tumoricidal activity of tumor necrosis factor-related apoptosis-inducing ligand in vivo. Nat. Med. 5, 157-163 (1999)) and NC1 domain of type X collagen (Zhang, Y. & Chen, Q., The noncollagenous domain 1 of type X collagen. A novel motif for trimer and higher order multimer formation without a triple helix. J. Biol. Chem. 274, 22409-22413 (1999)), can be used in the present invention.

The “SS-TFD”, constructed as described above, was fused to human TRAIL (amino acid 114-281) cDNA to construct the SS-TFD-TRAIL(amino acid 114-281) cDNA” cassette.

As the coding sequence for the rTRAIL protein, the sequence for the amino acid 114-281 region of TRAIL was used which was shown to induce apoptosis in many target cells including HeLa, MCF-7 and Jurkat (Pitti, R. M. et al., Induction of apoptosis by Apo-2 ligand, a new member of the tumor necrosis factor cytokine family. J. Biol. Chem. 271, 12687-12690 (1996)).

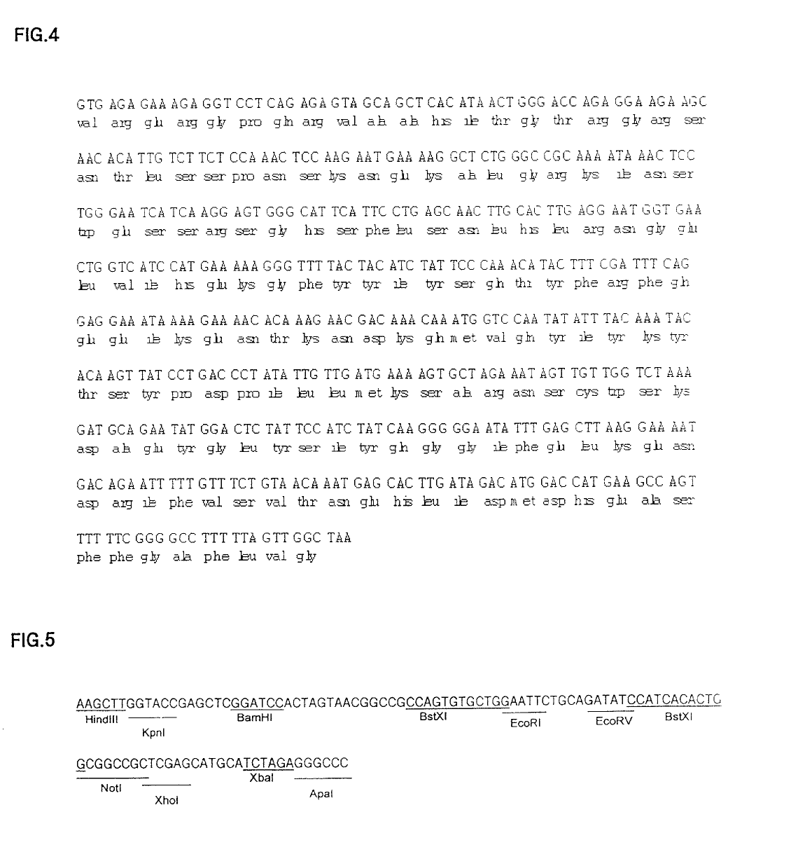

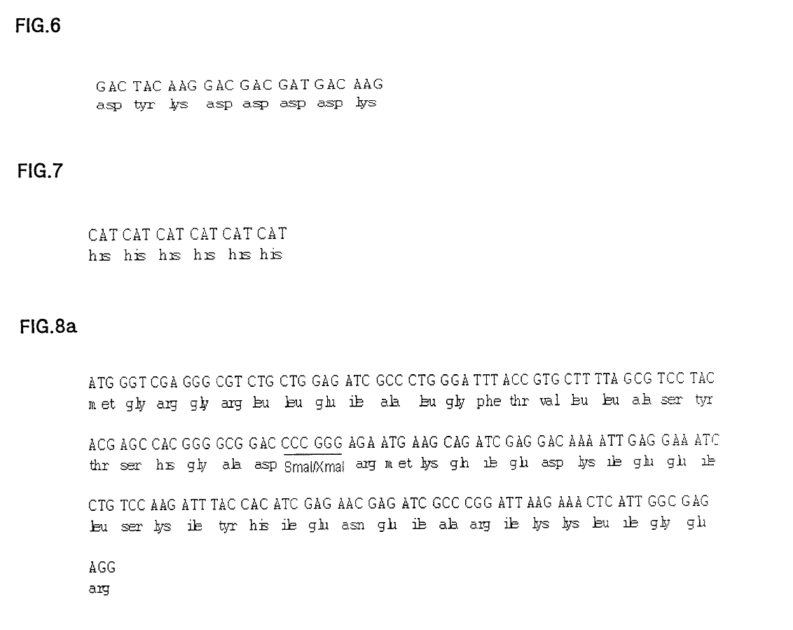

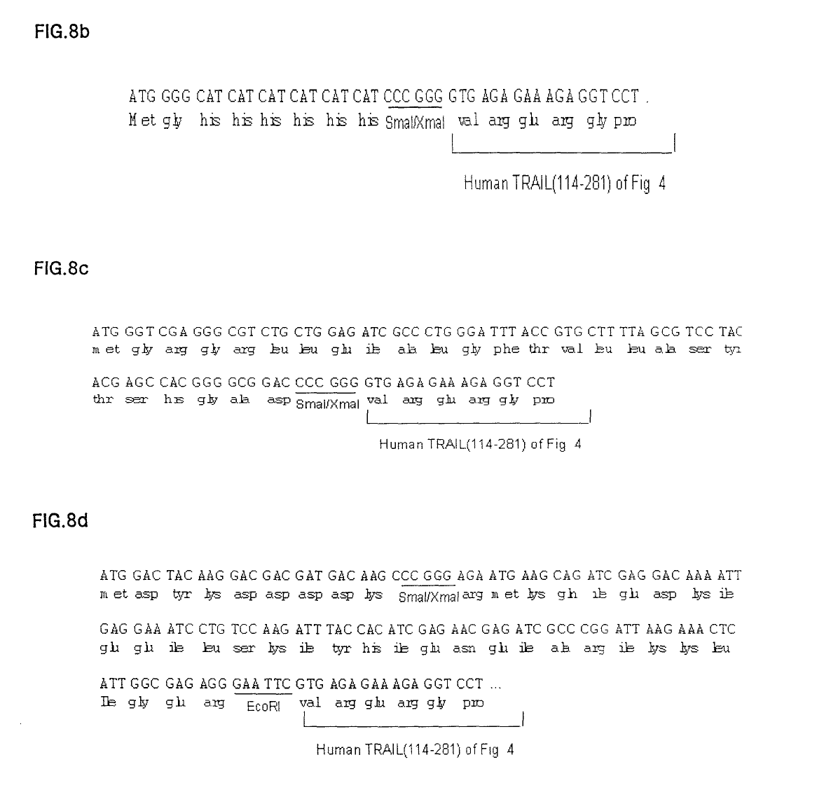

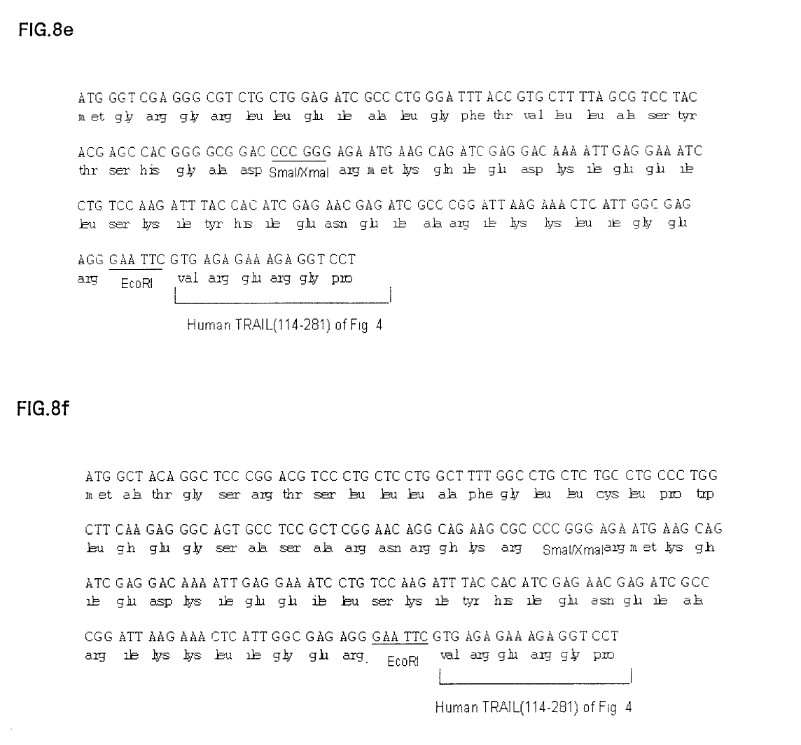

Human TRAIL (114-281) cDNA was generated by PCR using a primer set containing appropriate restriction sites at each end for facilitating subcloning. The resulting sequence is listed in FIG. 4.

Nucleotides can be changed by manipulations including point mutation, deletion or addition. However, the sequence is still considered as the human TRAIL and can be used in the present invention, if the amino acid sequence shows a sequence homology of at least 80% to the sequence in FIG. 4 and the biological function of the translated protein is not significantly different from TRAIL having the sequence in FIG. 4.

If a post-translational modification including glycosylation does not significantly affect the biological activity of the TRAIL(114-281) protein, the modified sequence can also be used in the present invention.

Nucleotides including restriction enzyme sites can be added to the 5′ or 3′ terminal of the human TRAIL(114-281) coding sequence.

Cells transfected with a recombinant expression vector harboring this “SS-TFD-TRAIL(114-281) cDNA” cassette produced secretable recombinant trimeric TRAIL (srtTRAIL) that is highly potent in target cell killing.

The TRAIL cassette of the present invention thus constructed can be cloned into an appropriate expression vector to be used in the large scale production of a secretable recombinant trimeric TRAIL protein or can be directly administered to a patient for gene therapy.

In one aspect, the present invention is characterized by mammalian expression vectors pCMVdw and pAAVdw, which can be used for the expression of foreign genes such as the TRAIL cassette described above.

The pCMVdw vector is a cytomegalovirus (CMV) promoter-driven mammalian expression vector. It is prepared by replacing the multiple cloning site (from HindII to ApaI) of the pCR3 vector (purchased from Invitrogen) with the sequence shown in FIG. 5.

The pCR3 vector is a vector belonging to the pcDNA series (Invitrogen) that is most widely used for the expression in mammalian cells, and is a plasmid harboring the early promoter of the cytomegalovirus (pCMV). pCMV is one of the most potent known promoters and known to induce a strong expression both in transient expression and in stable expression. This vector also has T7 and Sp6 promoter regions, enabling the in vitro transcription and translation using T7 or Sp6 polymerase. pCMV is also a high copy plasmid having the advantage that it facilitates vector preparation, and encodes a neomycin resistant gene such that it allows the establishment of a stable cell line or a stable expression of a foreign gene via transfection.

However, the multiple cloning site (MCS) of a pCR3 vector has a low utility since the various restriction enzyme sites included are not well localized or quite unique. To improve this, the pCMVdw vector useful for subcloning, was prepared by substituting the MCS with the sequence shown in FIG. 5b such that commonly used restriction enzyme sites are contained. Thus, the pCMVdw vector, which retains basic advantages of pCR3while having useful cloning sites, is useful for the mass production of TRAIL in mammalian cell lines and can be used for the constitutive expression of any foreign gene.

The pCMVdwSEC2 and pCMVdwSEC(CV) vectors, in which only secretion signals are integrated into the pCMVdw vectors can be used to express any foreign gene whose expression products require secretion for their biological functions. These vectors containing the foreign gene can be transiently or stably transfected into cultured cells to express the gene.

The pCMVdwILZ, in which the ILZ sequence is integrated into the pCMVdw vector, can be used to express any foreign gene whose expression products require trimerization for their biological functions. This vector containing the foreign gene can be transiently or stably transfected into cultured cells to express the gene.

The pCMVdwSEC2ILZ and pCMVdwSEC(CV)ILZ vectors, in which the secretion signals and the ILZ sequence is integrated into the pCMVdw vector, can be used to express any foreign gene whose expression products require secretion and trimerization for their biological functions. These vectors containing the foreign gene can be transiently or stably transfected into cultured cells to express the gene.

The constitutively active CMV and TK promoters of the AAV genome plasmid (pXX-UF1, obtained from Dr. X. Xiao, University of Pittsburgh) packaged into the recombinant AAV particles were replaced with a Tet-On inducible promoter (target gene expression is turned on by Tetracycline or its derivative Doxycycline) constructed by the present inventors to give an inducible expression vector pAAVdw.

In general, target genes can be expressed in two different manners, i.e., constitutive or inducible manner.

When the target gene is expressed in an inducible manner, it relies upon the stable expression wherein the target gene to be expressed is inserted into the genome. As systems for the inducible expression of target genes, there has been known Tetracycline/Doxycycline-inducible systems (Baron, U., Gossen, M. & Bujard, H., Tetracycline-controlled transcription in eukaryotes: novel transactivators with graded transactivation potential. Nucleic Acids Res. 25, 2723-2729 (1997)), ecdysone-inducible systems (No, D., Yao, T. P. & Evans, R. M., Ecdysone-inducible gene expression in mammalian cells and transgenic mice. Proc. Natl. Acad. Sci. USA 93, 3346-3351 (1996)), rapamycin-inducible systems (Rivera, V. M. et al., Long-term regulated expression of growth hormone in mice after intramuscular gene transfer. Proc. Natl. Acad. Sci. USA 96, 8657-8662 (1999)) and others. Among these, Tetracycline/Doxycycline-inducible systems are most widely used in vivo and have no serious side effects. Tetracycline and Doxycycline are cheap antibiotics that can be administered orally. Therefore, they are preferred over other systems in view of economic considerations.

Tetracycline/Doxycycline-inducible systems are largely composed of two parts.

The first part of the system expresses tTA or rtTA, and the expressed tTA or rtTA binds Tetracycline or Doxycycline. Most of the tTA or rtTA expression promoters developed thus far are fully functional CMV promoters. Therefore, tTA or rtTA is always expressed to a maximum allowed by the function of the CMV promoter.

The second part encodes a target gene that is to be regulated by the tTA or rtTA bound to Tetracycline or Doxycycline. The expression of the target gene is effected by a fused body of a minimal CMV promoter and an enhancer regulating the function of the minimal promoter instead of a fully functional CMV promoter. The function of the enhancer is controlled by the complex of tTA or rtTA and Tetracycline or Doxycycline. Basically, target gene expression is effected by the minimal CMV promoter. Thus, in the absence of the enhancer function, the basal expression level of the target gene is within a range allowed by the minimal CMV promoter.

In the choice of Tetracycline/Doxycycline-inducible systems, the systems are divided into Tet-On and Tet-Off depending on the type of TA (tTA or rtTA) which is the major element of the first part.

First, tTA can be chosen. tTA can act on the enhancer and thus perform the enhancing function only in the non-bound state where it is not bound to Tetracycline or Doxycycline. However, when tTA forms a complex with Tetracycline or Doxycycline, the complex loses the function of acting on the enhancer and thus cannot perform the enhancing function any longer. Therefore, when tTA is included in a Tetracycline/Doxycycline system, so-called Tet-Off system is established which stops the target gene expression in the presence of Tetracycline or Doxycycline. If this system is selected, there comes the disadvantage that, when the physiological activity of the target gene per se is used in the treatment of diseases, a patient should take Tetracycline or Doxycycline for the whole period where the expression of the target gene is not required, due to the continued expression of the target gene after the termination of the treatment.

When rtTA is selected, in contrast to tTA, it can act on the enhancer and thus perform the enhancing function only in the bound state where it is bound to Tetracycline or Doxycycline. However, when rtTA fails to form a complex with Tetracycline or Doxycycline, it loses the function of acting on the enhancer and thus cannot perform the enhancing function any longer. Therefore, when rtTA is included in a Tetracycline/Doxycycline system, so-called Tet-On system is established which allows the target gene expression only in the presence of Tetracycline or Doxycycline. If this system is selected, when the physiological activity of the target gene per se is used in the treatment of diseases, the treatment is initiated only with the administration of Tetracycline or Doxycycline. When the treatment is terminated, patients can stop the expression of the target gene by quitting the administration of Tetracycline or Doxycycline. This suggests that the Tet-On system is far more advantageous than the Tet-Off system.

In view of this advantage, a Tet-On system is chosen in the present invention. Furthermore, the present inventors have constructed a new Tet-On system such that considerable operational advantages are provided compared with known Tet-On systems.

Since the promoter for expressing rtTA in known Tet-On systems is a full length CMV promoter, rtTA was always strongly expressed. In contrast, the present invention uses a minimal CMV promoter instead of a full length CMV promoter, leading to the minimization of the basal expression level. This system was designed to operate by a feed-forward amplification loop such that a stronger expression is achieved once the expression has been induced, because the minimal promoter used in the present invention binds to an enhancer that is regulated by the complex of rtTA and Tetracycline or Doxycycline. That is, minimally expressed rtTA forms complex with Tetracycline or Doxycycline in the presence of them, and the complex binds to the enhancer resulting in a drastic increase in the minimal CMV promoter function. The increased promoter function amounts to about 5 times the activity of a full length CMV promoter. In a TetOn system, the expression of the target gene is directly proportional to the degree of rtTA expression in the presence of Tetracycline or Doxycycline. Thus, the Tet-On system of the present invention would be considered significantly superior to known Tet-On systems in terms of the ultimate level of expression. The pAAVdw vector enables the maximum expression of target genes by expressing rtTA by a feed-forward amplification loop. That is, in the presence of inducing factors Tetracycline or Doxycycline, the target gene expression is realized in the form of a continued amplification over time, leading the target gene expression to reach the maximum possible.

It should be noted that, as opposed to known Tet-On systems where rtTA is always strongly expressed, the rtTA expression in the Tet-On system of the present invention is regulatable. If a known Tet-On system is used as part of a therapy, the possibility that the strong constant expression of rtTA may cause an immune response against rtTA cannot be neglected. The Tet-On system of the present invention has the advantage that, even if rtTA inevitably causes an immune response, the degree of the response can be minimized.

When a target gene is expressed under the Tet-On system of the pAAVdw vector of the present invention, a strong expression of the target gene can be induced economically and safely using Tetracycline or Doxycycline which is a cheap antibiotic capable of oral administration.

In transient transfection experiments using pAAVdwGFP vectors containing gfp, a gene encoding a green fluorescent protein, as the target gene, target gene (gfp) expression was found to be highly inducible in response to Tetracycline.

When pAAVdwSEC2ILZTRAIL or pAAVdwSEC(CV)ILZTRAIL, wherein the gfp gene in the pAAVdwGFP was replaced by a TRAIL DNA cassette of the present invention encoding a secretable trimeric TRAIL, was used to transiently transfect cultured cells, they produced and secreted a trimeric rTRAIL in response to Tetracycline and exhibited a potent killing effect on cancer cells.

The recombinant vector pAAVdwSEC2ILZTRAIL(14-281) and pAAVdwSEC(CV)ILZTRAIL(114-281) containing the TRAIL cassette of the present invention can be transiently or stably introduced into biological systems including cultured cells to produce biologically active rTRAIL protein, by various methods such as the Calcium phosphate precipitation method, liposome-mediated transfection, and virusmediated transduction (Wolff, J. A., Gene therapeutics. Wolff, J. A (Eds.), Birkhauser Boston press, Cambridge, Mass. (1994)).

In addition, the pAAVdwSEC2ILZTRAIL(114-281) and pAAVdwSEC(CV)ILZTRAIL(114-281) vectors can be used to produce AAV particles using a conventional production method of adeno-associated virus (Samulski, R. J., Chang, L. S. & Shenk, T., Helper-free stocks of recombinant adeno-associated viruses: normal integration does not require viral gene expression. J. Virol. 63, 3822-3828 (1989)) or a helper plasmid-based Adenovirus-free method (Xiao, X., Li, J. & Samulski, R. J., Production of high-titer recombinant adeno-associated virus vectors in the absence of helper adenovirus. J. Virol. 72, 2224-2232 (1998); Grimm, D., Kern, A., Rittner, K. & Kleinschmidt, J. A., Novel tools for production and purification of recombinant adenoassociated virus vectors. Hum. Gene Ther. 9, 2745-2760 (1998)).

One aspect of the present invention is characterized by the systemic or intramuscular administration of the pCMVdw or pAAVdw vectors containing the TRAIL cassette in the form of an injectable preparation for the treatment of diseases including cancer.

The pCMVdw vector expressing an rTRAIL constitutively can be administered into the body whenever necessary. The pAAVdw vector expressing an rTRAIL inducibly can also be administered into the body whenever necessary by the systemic or intramuscular route. In this case, the expression of the rTRAIL gene is induced by Tetracycline or Doxycycline when the patient takes the inducing factor, Tetracycline or Doxycycline, resulting in the production and secretion into the circulation of TRAIL in vivo.

Another aspect of the present invention is characterized by a gene therapy using AAV particles packaged from a recombinant pAAVdw vector containing the rTRAIL expression cassette.

Since AAV particles are known to transduce muscle cells very well, they are primarily administered in the form of an intramuscular injection when used in a gene therapy. When AAV particles containing the rTRAIL cassette of the present invention are used as medicine, they can also be administered in the form of an intramuscular injection. The suitable number of AAV particles to be administered is 1012 per kg bodyweight of the patient. The Tetracycline/Doxycycline-inducible rTRAIL expression cassette in the administered AAV particles will be incorporated into the human genome, allowing a stable and long-term expression of rTRAIL. Consequently, after a single administration to patients, the patients can effect the rTRAIL expression in their body almost permanently by taking Tetracycline or Doxycycline. Because Tetracycline and Doxycycline are cheap antibiotics that can be taken orally, patients can endogenously produce rTRAIL whenever necessary simply by taking a cheap inducing factor. This means that patients can receive treatments economically and effectively over an extended period of time.

Such pAAVdw vectors can be used for the inducible expression of any other gene, and if the gene encodes a protein for the treatment of a disease, a pAAVdw vector harboring the gene as the target gene and virus particles packaged from this vector can also be used for a gene therapy of the disease at issue. Thus, the treatment of diseases using such pAAVdw vectors constitutes another aspect of the present invention.

In addition, for the expression of the DNA cassette of the present invention containing SEC2ILZTRAIL(114-281) or SEC(CV)ILZTRAIL(114-281), any plasmid expression vector or viral expression vector (e.g., Adenovirus, Adeno-associated virus, retrovirus and Herpes virus) harboring any promoter (e.g., simianvirus (SV) 40 promoter, long terminal repeat (LTR) of the rous sarcoma virus (RSV)) that can substitute the function of a CMV promoter, as well as the pCMVdw and pAAVdw vectors of the present invention, can be utilized and constitute a further aspect of the present invention.

The recombinant expression vectors containing SEC2ILZTRAIL(114-281) and SEC(CV)ILZTRAIL(114-281) can be delivered to biological systems including cells by various methods such as the Calcium phosphate precipitation method, liposome-mediated transfection, and virus-mediated transduction (Wolff, J. A., Gene therapeutics. Wolff, J. A (Eds.), Birkhauser Boston press, Cambridge, Mass. (1994)).

The pAAVdwSEC2 and pAAVdwSEC(CV) vectors, in which only secretion signals are integrated into the pAAVdw vectors, can be used to express any foreign gene whose expression products require secretion for their biological functions. These vectors containing the foreign gene can also be transiently or stably transfected into cultured cells to express the gene. These vectors containing foreign genes can be packaged into AAV particles.

The pAAVdwILZ, in which the ILZ sequence is integrated into the pAAVdw vector, can be used to express any foreign gene whose expression products require trimerization for their biological functions. This vector containing the foreign gene can also be transiently or stably transfected into cultured cells to express the gene. This vector containing foreign genes can be packaged into AAV particles.

The pAAVdwSEC2ILZ and pAAVdwSEC(CV)ILZ vectors, in which the secretion signals and the ILZ sequence are integrated into the pAAVdw vector, can be used to express any foreign gene whose expression products require secretion and trimerization for their biological functions. These vectors containing the foreign gene can also be transiently or stably transfected into cultured cells to express the gene. These vectors containing foreign genes can be packaged into AAV particles.

The present invention further provides a pharmaceutical composition for the treatment of cancer, comprising the TRAIL protein of the present invention and ActD.

In general, combination therapies produce a better prognosis in the cancer treatment than individual therapies. Many chemotherapeutic agents are known to cause cytotoxic side effects at an effective dosage. If a low dosage of a chemotherapeutic agent can fortify rTRAIL (especially against TRAIL-resistant cancers), this combination therapy would be superior to rTRAIL alone. A chemotherapeutic agent Actinomycin D (ActD) greatly enhanced TRAIL-induced apoptosis in various cancer cell lines. ActD is known to inhibit the function of RNA polymerase II, resulting in blockade of mRNA synthesis. Enhancing activity of ActD was obtained at a level of 100 ng/ml, a concentration that is 1/10 of the dosage commonly used to block mRNA synthesis. ActD induced p53 expression dramatically, but had no effect on signaling components involved in TRAIL action (Seol, D. W., Seol, M. H. & Billiar, T. R., Tumor necrosis factor-related apoptosis-inducing ligand (TRAIL) induces apoptosis independent of p53 status. Manuscript in preparation). Regardless of p53 status, which is considered a critical factor in cancer therapies using chemotherapeutic agents or radiation, rTRAIL/ActD killed target cells effectively. Furthermore, it was found that many cancer cell lines resistant to rTRAIL alone can be effectively killed by rTRAIL/ActD. These results are very significant for cancer therapy since it is well known that more than 50% of human cancers contain various p53 mutations (Vogelstein, B. & Kinzler, K. W. a., p53 function and dysfunction. Cell 70, 523-526 (1992); Hollstein, M., Sidransky, D., Vogelstein, B. & Harris, C. C., p53 mutations in human cancers. Science 253, 49-53 (1991)). Considering the limited cytotoxic side effects of rTRAIL in vivo, rTRAIL fortified by low dose ActD may become an improved cancer therapy.

ActD can be used not only with a recombinant human TRAIL(114-281) protein, but also with any plasmid expressing a recombinant human TRAIL(114-281) protein including pCMVdwSEC2ILZTRAIL(114-281), pCMVdwSEC(CV)ILZTRAIL(114-281), pAAVdwSEC2ILZTRAIL(114-281) and pAAVdwSEC(CV)ILZTRAIL(114-281).

ActD can be used with AAV particles generated using pAAVdwSEC2ILZTRAIL(114-281) and pAAVdwSEC(CV)ILZTRAIL(114-281).

ActD can also be replaced with other chemotherapeutic agents including anticancer drugs that enhance the apoptotic activity of recombinant human TRAIL(114-281).

The present invention will now be described in detail by Examples. It should be appreciated that the following Examples are given for the purpose of illustration only and should not be construed as limiting the scope of the present invention that is given in the claims.

### EXAMPLES

### Example 1

Construction of a SS-TFD-TRAIL(amino acid 114-281) cassette The SEC2 signal sequence (FIG. 1a) and the SEC(CV) signal sequence (FIG. 1b) were chemically synthesized. The KpnI (5′) and Sma XmaI (3′) sites were added to the respective ends of the sequences to facilitate cloning. Each signal sequence was inserted into the pCMVdw vector (FIG. 5) that had been digested with KpnI and XmaI to construct the pCMVdwSEC2 and pCMVdwSEC(CV) vectors.

The second component, the trimer-forming domain ILZ sequence (FIG. 3) was also chemically synthesized and SmaI/XmaI (5′) and EcoRI (3′) sites were added to the respective ends of the sequence to facilitate cloning. The previously formed pCMVdwSEC2 and pCMVdwSEC(CV) vectors were digested with XmaI and EcoRI and the newly synthesized ILZ sequence was inserted into the digested sites to construct the pCMVdwSEC2ILZ and pCMVdwSEC(CV)ILZ vectors.

A cDNA sequence encoding the human TRAIL(114-281) protein was prepared as follows. First, the open reading frame (ORF) of human TRAIL was obtained from a PCR using a human hepatocyte cDNA library (Invitrogen) as templates by using a primer set (sense: 5′-CCCGGTACCATGGCTATGATGGAGGTCCAGGGG-3′, antisense: 5′CCCGAATTCTTAGCCAACTAAAAAGGCCCCGAA-3′). The PCR was carried out under the conditions that after 5 min. of template denaturation at 95° C., 35 cycles of 1 min. at 95 ° C., 1 min. at 55 ° C., and 1 min. at 72° C. were repeated. The PCR product was digested with KpnI and EcoRI and purified and isolated after an electrophoresis on an agarose gel. The purified fraction was ligated to a pCMVdw vector digested with KpnI and EcoRI to construct pCMVdwTRAIL. A cDNA sequence encoding human TRAIL(114-281) protein was generated by PCR using the pCMVdwTRAIL as template and using a primer set (sense: 5′-CCCGAATTCGTGAGAGAAAGAGGTCCTCAGAG-3′, antisense: 5′-CGTAGCGGCCGCTTAGCCAACTAAAAAGGCCCCGAAA-3′). The PCR was carried out under the conditions that after 5 min. of template denaturation at 95° C., 35 cycles of 1 min. at 95 ° C., 1 min. at 55 ° C., and 1 min. at 72 ° C. were repeated. The PCR product was digested with EcoRI(5′) and NotI(3′) and inserted into the EcoRI(5′) and NotI(3′) sites of pCMVdwSEC2ILZ and pCMVdwSEC(CV)ILZ vectors to construct pCMVdwSEC2ILZTRAIL(114-281) and pCMVdwSEC(CV)ILZIRAIL(114-281), producing a TRAIL cassette constructed in vectors.

The resulting recombinant vector pCMVdwSEC2ILZTRAIL(114-281) was deposited on Jun. 9, 2000 under the Deposit Number PTA-2018, and pCMVdwSEC(CV)ILZTRAIL(114-281) was deposited on Jun. 9, 2000 under the Deposit Number PTA-2019.

### Example 2

Construction of recombinant expression vectors pCMVdwSEC2ILZTRAIL(114-281) and pCMVdwSEC(CV)ILZTRAIL(114-281) containing the TRAIL cassette and examination of their effects.

In order to examine the TRAIL production and secretion by the TRAIL cassette, the multiple cloning site (from HindIII to ApaI) of the pCR3 vector (Invitrogen) was replaced with the sequence shown in FIG. 5 to give pCMVdw, a constitutive expression vector.

pCR3 vector was first digested with HindIII and ApaI, and the vector fraction was purified and isolated by agarose gel electrophoresis. The sequence shown in FIG. 5, synthesized chemically, was then ligated to the purified and isolated pCR3 vector to construct the pCMVdw vector.

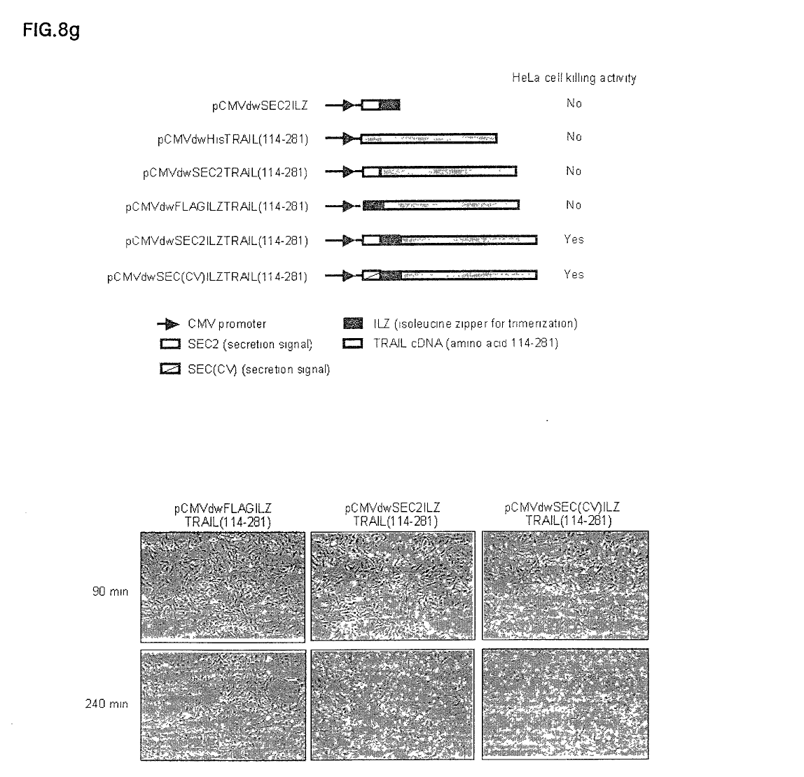

Various genes were cloned into the resulting pCMVdw vector to prepare the various recombinant vectors having the structures shown in FIG. 8g.

The SEC2 sequence (FIGS. 8a, 8c, 8e and 8g) were cloned into the KpnI(5′) and SmaI/XamI(3′) sites of the pCMVdw vector.

The His-tag sequence (FIGS. 8b and 8g) containing two additional amino acids (Met and Gly) at its N-terminus was cloned into the KpnI(5′) and SmaI/XmaI(3′) sites of the pCMVdw vector. The His-tag sequence has a high affinity to divalent cations such as Ni and Co and, when fused to the N-terminus or C-terminus of a protein, facilitates the affinity purification of the protein by affinity resins. In the present invention, the His-tag sequence was used for providing the translation start site.

The FLAG-tag sequence (FIGS. 8d and 8g) containing an additional amino acid (Met) at its N-terminus was cloned into the KpnI(5′) and SmaI/Xmal(3′) sites of the pCMVdw vector. The FLAG-tag sequence is specifically recognized by anti-FLAG antibodies and, when fused to a protein, facilitates the affinity purification of the protein by FLAG antibodies. When a specific antibody against a target protein is not available, the detection of the expressed protein can be carried out using anti-FLAG antibodies if the FLAG sequence is fused to the target protein. In the present invention, the FLAG-tag sequence was used for providing the translation start site.

The SEC(CV) sequence (FIGS. 8f and 8g) was cloned into the KpnI(5′) and SmaI/XmaI(3′) sites of the pCMVdw vector.

The ILZ sequence was fused to the SEC2 sequence (FIGS. 8a, 8e and 8g), to the FLAG-tag sequence (FIGS. 8d and 8g) or to the SEC(CV) sequence (FIGS. 8f and 8g) by inserting it into the SmaI/XmaI(5′) and EcoRI(3′) sites.

The human TRAIL(114-281) cDNA was fused to the His-tag sequence (FIGS. 8b and 8g), to the SEC2 sequence (FIGS. 8c and 8g) or to the FLAGILZ sequence (FIGS. 8d and 8g) by inserting it into the Smal/XmaI(5′) and EcoRI(3′) sites, and to the SEC2ILZ sequence (FIGS. 8e and 8g) or to the SEC(CV)ILZ sequence (FIGS. 8f and 8g) by inserting it into the EcoRI(5′) and NotI(3′) sites.

The resulting recombinant vectors are shown in FIG. 8g.

The expression vectors thus constructed were examined for their HeLa cell killing activity. Transfection was carried out using the calcium phosphate method or liposomes. The Calcium phosphate method was performed with CellPhect transfection kit (Pharmacia) or using standard procedures (Sambrook, J., Fritsch, E. F. & Maniatis, T., Molecular cloning: a laboratory manual, 2nd ed. Cold Spring Harbor Laboratory press, Cold Spring Harbor, N.Y. (1989)), and the liposomes used were GenePORTER (Gene Therapy System) or LipofectAMINE (Life Technologies). Comparable results were produced in all processes used.

To perform the experiment illustrated in FIG. 8g, each plasmid (2 μg) was transiently transfected into 293 cells, a TRAIL-resistant cell line, using the Calcium phosphate method. 36 hours after transfection, the individual culture medium was saved for the HeLa cell killing activity test. HeLa cells plated in 6 wells were incubated with the test culture medium for 4 hours. Only the culture medium obtained by the transfection of the pCMVdwSEC2ILZTRAIL(114-281) or pCMVdwSEC(CV)ILZTRAIL(114-281) killed HeLa cells effectively. Cell viability assay using crystal violet (Seol, D. W. & Billiar, T. R., A caspase-9 variant missing the catalytic site is an endogenous inhibitor of apoptosis. J. Biol. Chem. 274, 2072-2076 (1999)) or by conventional viable cell counting under the microscope (Pan, G. et al., The receptor for the cytotoxic ligand TRAIL. Science 276, 111-113 (1997); Pan, G. et al., An antagonist decoy receptor and a death domain-containing receptor for TRAIL. Science 277, 815-818 (1997)) revealed that 50% (pCMVdwSEC2ILZTRAIL(114-281)) and 90% (pCMVdwSEC(CV)ILZTRAIL(114-281)) of HeLa cells were killed within four hours.

This transient transfection and functional tests (FIG. 8g) clearly indicate that the TRAIL DNA cassette of the present invention is capable of producing a secretable TRAIL protein that can be secreted and effectively kill HeLa cells.

### Example 3

Test for the expression, secretion and cleavage of TRAIL protein.

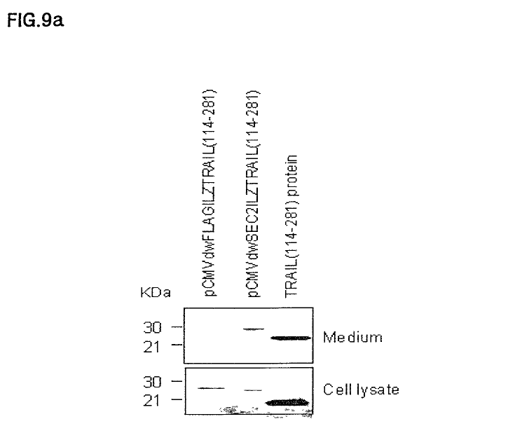

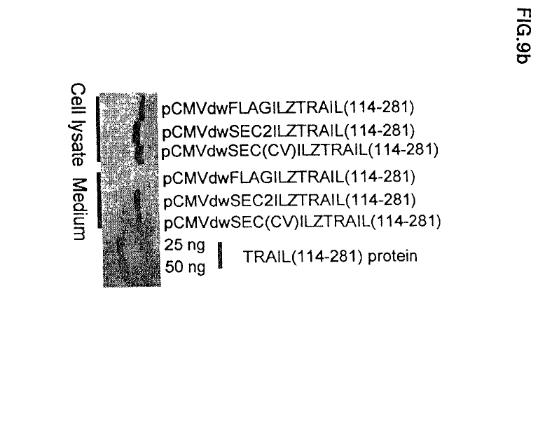

Expression, secretion and specific cleavage of TRAIL(114-281) protein were examined. SEC2ILZTRAIL(114-281) protein containing the SEC2 secretion signal and SEC(CV)ILZTRAIL(114-281) protein containing the SEC(CV) secretion signal were secreted into the culture medium, but the FLAGILZTRAIL(114-281) protein was not (FIGS. 9a and 9b).

The FLAGILZTRAIL(114-281) protein was detectable in the whole cell lysate only (FIG. 9b), which suggests that a secretion signal is required for the secretion and biological functioning of the TRAIL(114-281) protein.

The secreted ILZTRAIL(114-281) protein produced by pCMVdwSEC(CV)ILZTRAIL(114-281) was identified to be smaller than cytosolic ILZTRAIL(114-281) protein. This result indicates that a specific Furin-mediated cleavage occurred.

The “TRAIL(114-281) protein” in FIGS. 9a and 9b is a recombinant protein purified from bacterial cells harboring a known bacterial expression plasmid (Seol, D. W. & Billiar, T. R., A caspase-9 variant missing the catalytic site is an endogenous inhibitor of apoptosis. J. Biol. Chem. 274, 2072-2076 (1999); Seol, D. W. & Billiar, T. R., Cysteine 230 modulates tumor necrosis factor-related apoptosis-inducing ligand activity. Cancer Res. 60, 3152-3154 (2000)). The protein was used as a positive control for TRAIL antibodies.

All TRAIL(114-281) proteins with or without the fused accessory sequences (FLAG-ILZ or SEC2ILZ) were detected by standard Western Blotting assays.

Human ILZTRAIL(114-281) protein can be produced by transient or stable transfection of the DNA sequence “SEC2ILZTRAIL(114-281)” or “SEC(CV)ILZTRAIL(114-281)”. The secreted or cytosolic ILZ-fused TRAIL(114-281) protein can be purified by various methods including the conventional column purification. “SEC2ILZTRAIL(114-281)” or “SEC(CV)ILZTRAIL(114-281)” can also be fused to additional affinity tag sequences such as HA-tag or Myc-tag for affinity purification. If purified ILZTRAIL(114-281) protein has the biological functions of original human TRAIL(114-281) protein, the protein belongs to the present invention.

### Example 4

Preparation and Activity Test of pAAVdw Vector.

pAAVdw vector was prepared which can inducibly express an inserted target gene by the Tet-On system of feed-forward amplification loop.

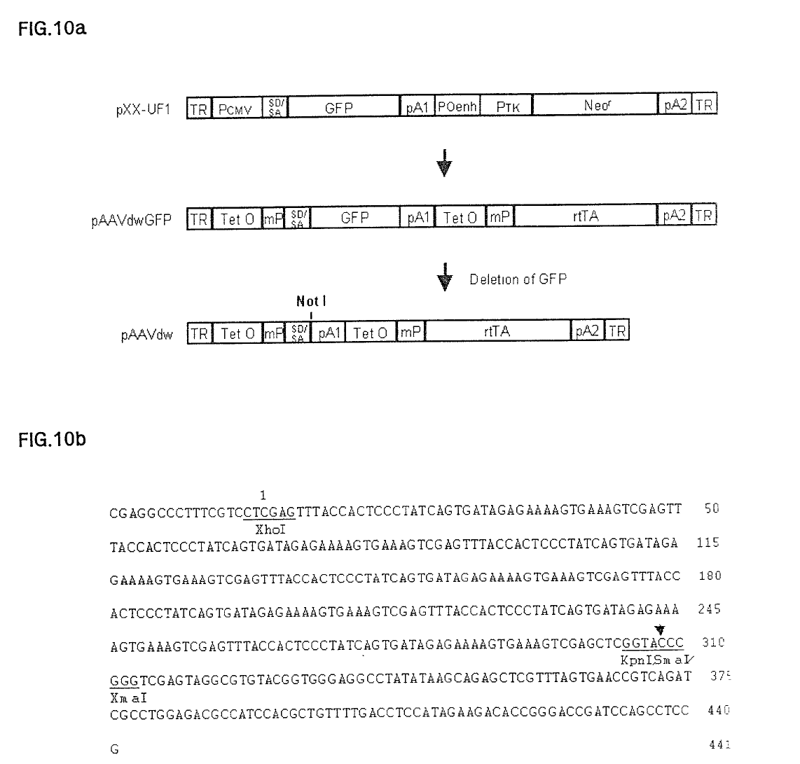

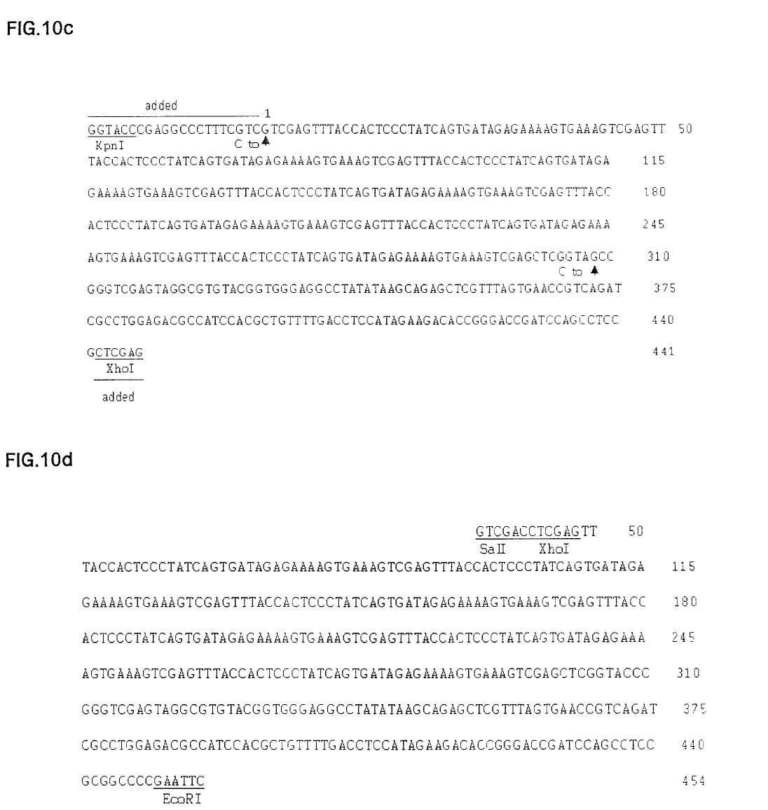

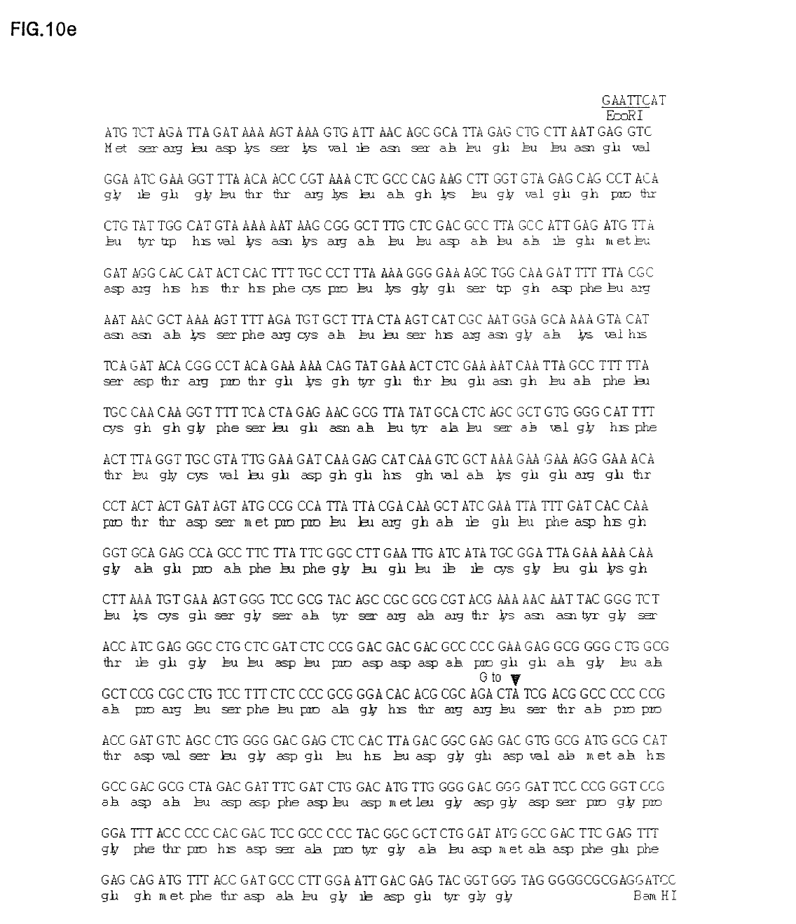

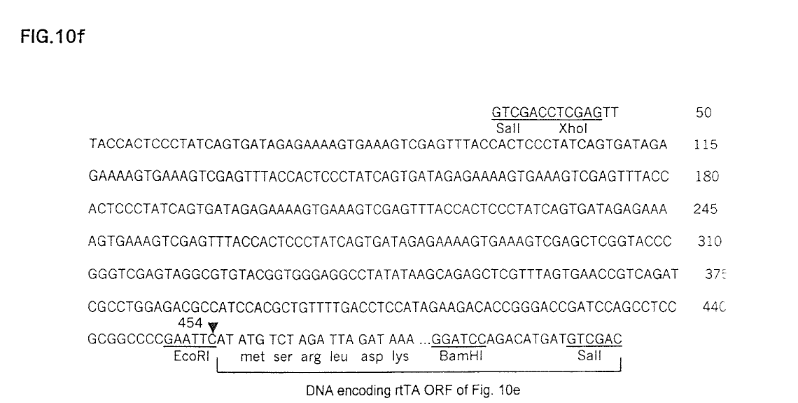

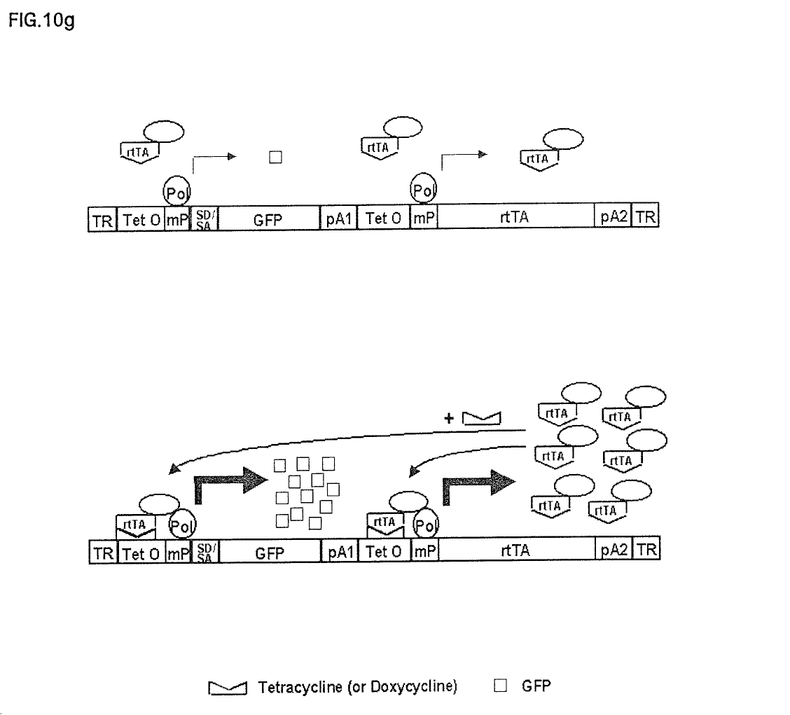

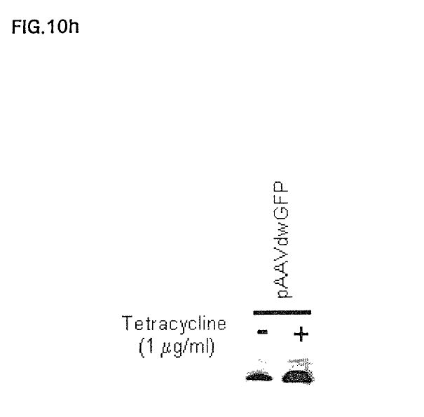

To prepare the pAAVdwGFP vector prior to the preparation of pAAVdw vector, the “Poenh-PTK-Neor” region was removed from the pXX-UF1 vector (FIG. 10a) by cutting the pXX-UFl with SalI to construct the “pXX-UF1ΔSalI” vector. The “pXX-UF1ΔSalI” vector was further cut with KpnI and XhoI to delete the PCMV region (FIG. 10a).

The 308th nucleotide “C” (FIG. 10b) of pTRE plasmid (Clontech) containing the tetracycline response element (TetO) and the CMV minimal promoter (mP) (FIGS. 10a and 10b) was mutated to “G” (FIG. 10c) using a primer set (sense: 5′GTCGAGCTCGGTAGCCGGGTCGAGTAG-3′, antisense: 5′CTACTCGACCCGGCTACCGAGCTCGAC-3′, mutated nucleotide underlined) by Quick Change point mutation method (Stratagen). This point mutation simultaneously knocked out the internal KpnI and SmaI/XmaI sites. Using a primer set (sense: 5′CCCGGTACCCGAGGCCCTTTCGTCGTCGAGTTTACC-3′, mutated nucleotide underlined, antisense: 5′-CCCCTCGAGCGGAGGCTGGATCGGTCCCGGTG-3′) and the mutant pTRE plasmid as a template, PCR was carried out to prepare the left-arm “TetO-mP” sequence (FIG. 10c). The internal XhoI restriction site was mutated by the 5′ PCR primer in which the 1st nucleotide “C” was changed to “G” during its chemical synthesis. This PCR product was cut with KpnI and XhoI, purified and ligated to the KpnI/XhoI-cut “pXX-UF1ΔSalI” vector to construct the “pXXUF1ΔSalI/TetO-mP” vector.

To prepare the DNA sequence encoding the right-arm “TetO-mP-rtTA” of the pAAVdw vector, the DNA harboring the rtTA ORF (FIG. 10e) was isolated from pTet-On plasmid (Clontech) by cutting the plasmid with EcoRI and BamHil. This rtTA ORF was inserted into the pTRE plasmid containing the TetO-mP by replacing the EcoRI-BamHI region, resulting in the “pTRE-rtTA” vector. The “TetO-mP-rtTA” region of the pTRErtTA vector was amplified by PCR using a SalI-containing primer set (sense: 5′CCCGTCGACCTCGAGTTTACCACTCCCTATCAG-3′, antisense: 5′CCCGTCGACATCATGTCTGGATCCTCGCGCCCC-3′), blunted and ligated to the Smal-cut pGEX-2T vector (Pharmacia) to construct the “pGEX-2T/TetO-mP-rtTA” plasmid. After mutating the internal SalI restriction site in rtTA using a primer set (sense: 5′-CACACGCGCAGACTATCGACGGCCCCCCCG-3′, antisense: 5′CGGGGGGGCCGTCGATAGTCTGCGCGTGTG-3′, mutated nucleotide underlined) (arrow, FIG. 10e), the DNA (FIG. 10f) containing the “TetO-mP-rtTA” was cut with SalI, purified and ligated to the SalI-cut “pXXUF1ΔSalI/TetO-mP” vector to construct the “pAAVdwGFP” vector. Then the pAAVdwGFP vector was cut with NotI to remove the GFP region and self-ligated to construct the pAAVdw vector. The resulting pAAVdw vector was deposited to ATCC under the Access Number PTA-2102 on June 20, 2000, in the form of pAAVdwGFP containing the gfp gene.

The left-arm TetO contains seven repeats (FIG. 10c) of tetracycline response element (TRE), whereas the right-arm TetO contains six repeats (FIG. 10d) of TRE.

The pAAVdw vector was designed to be inducible in response to Tetracycline/Doxycycline. In this vector system, target gene expression is regulated by a feed-forward amplification loop (FIG. 10g). A CMV minimal promoter (mP) expresses a basal level of a target gene (i.e., gfp in the pAAVdwGFP) and rtTA protein that is a fusion protein composed of TetO binding domain and a transcription activation domain. In the absence of Tetracycline/Doxycycline, rtTA protein cannot bind to the TetO enhancer region. In contrast, Tetracycline or Doxycycline complexed with rtTA protein induces minimally existing rtTA protein to bind the TetO and activate the transcription machinery. As a result, more rtTA protein is expressed. This feed-forward amplification loop can make possible maximum expression of the target gene.

The target gene expression and Tetracycline-inducibility by the pAAVdw vector was tested using a gfp gene. To do this, 2 μg of the pAAVdwGFP vector was transiently transfected into 293 cells plated in 6 wells. 24 hours after transfection, Tetracycline (1 μg/ml) was added to the culture medium and incubated for an additional 24 hours. An aliquot of the culture medium (30 μl) was removed, mixed with loading buffer, boiled and loaded onto an SDS gel. The resolved proteins were transferred onto a nitrocellulose membrane and subjected to Western blotting assays using GFP-recognizing antibody. The result clearly indicates that target gene expression is highly induced by Tetracycline (FIG. 10h). It also implies that any other gene replacing the gfp gene can be expressed like gfp by the pAAVdw vector in a Tetracycline-inducible manner.

### Example 5

Expression of Recombinant TRAIL(114-281) Protein Using the pAAVdw Vector

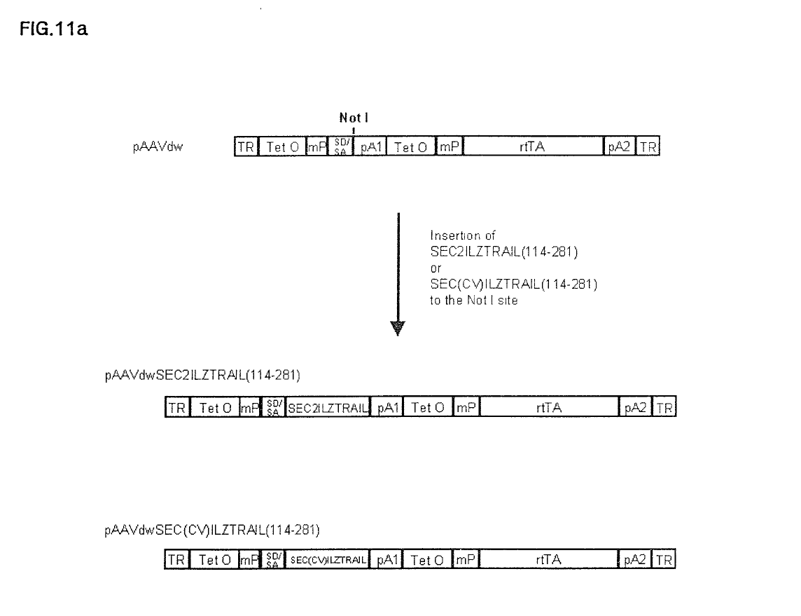

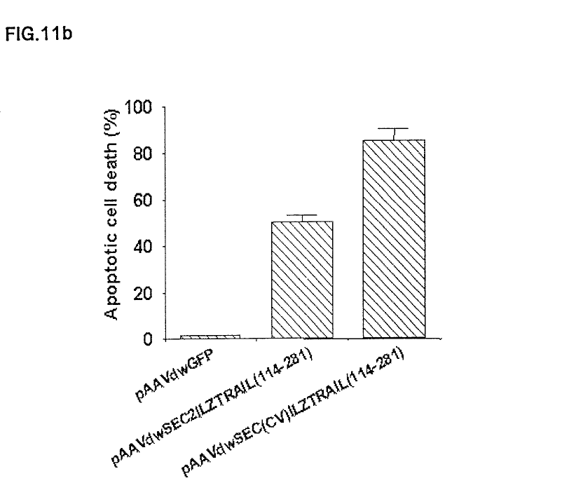

To express recombinant TRAIL(114-281) protein using the pAAVdw vector, DNA sequences for SEC2ILZTRAIL(114-281) and SEC(CV)ILZTRAIL(114-281) were prepared by PCR using a primer set (sense: 5′CCTAGCGGCCGCACTAGAGAACCCACTGCTTACTGG-3′, antisense: 5?CGTAGCGGCCGCTTAGCCAACTAAAAAGGCCCCGAAA-3′) and pCMVdwSEC2ILZTRAIL(114-281) and pCMVdwSEC2ILZTRAIL(114-281) as templates, respectively, and cloned into the NotI site in pAAVdw vector (FIG. 11a).

To test the functional activity of the proteins expressed by the pAAVdwSEC2ILZTRAIL(114-281) and pAAVdwSEC(CV)ILZTRAIL(114-281), 2 μg of each plasmid was transiently transfected into 293 cells plated in 6 wells using the Calcium phosphate method. 24 hours after transfection, Tetracycline (1 μg/ml) was added to the culture medium and incubated for an additional 24 hours. The culture media were then saved for the tests of apoptotic activity in HeLa cells. HeLa cells were plated in 12 wells. 24 hours after plating, the culture media of HeLa cells were replaced with the test culture media (1 ml). After 4 hours of incubation, dead cells with typical apoptotic morphology were counted under a microscope, or the cell viability was measured by a spectrophotometric analysis using crystal violet. The results clearly indicate that both SEC2ILZTRAIL(114-281) and SEC(CV)ILZTRAIL(114-281) proteins expressed by pAAVdw vector have apoptotic activity comparable with those expressed by the pCMVdw vectors (FIG. 1b). These results imply that the expression of SEC2ILZTRAIL(114-281) and SEC(CV)ILZTRAIL(114-281) is successfully induced by Tetracycline and that the proteins are secreted into the culture medium, as was shown in the case of GFP expression.

### Example 6

Virus Packaging and Test for the Therapeutic Effects

The Tetracycline/Doxycycline-inducible expression vector pAAVdw containing the DNA sequence for GFP, SEC2ILZTRAIL(114-281) or SEC(CV)ILZTRAIL(114-281) was packaged into AAV particles using a transient transfection method aided by a helper plasmid.

pAAVdwGFP (2 μg), pAAVdwSEC2ILZTRAIL(114-281) (2 μg) or pAAVdwSEC(CV)ILZTRAIL(114-281) (2 μg) vectors were transiently transfected into 293 cells plated in 6 wells along with a helper plasmid pDG vector (Grimm, D., Kern, A., Rittner, K. & Kleinschmidt, J. A. Novel tools for production and purification of recombinant adenoassociated virus vectors. Hum. Gene Ther. 9, 2745-2760 (1998)) (6 μg) using the Calcium phosphate method. 48 hours after transfection, the culture media were removed and the transfected 293 cells were collected. The cells were then resuspended in 400 μl of PBS and disrupted by repeated freeze-thawing (3 times).

The unpurified whole cell lysate was centrifuged at 12,000 rpm for 5 min. and the supernatant containing the packaged AAV particles was recovered and subjected to an inducibility test for Tetracycline as well as a test of therapeutic effects.

To 293 cells in 6 wells showing about 50% confluency, 400 μl of a supernatant containing the packaged AAV particles was added in two portions, allowing the introduction of AAV particles into the target cells. After 6 hours, the target cell culture medium containing the AAV particles was removed, and incubated with fresh target cell culture medium for 12 hours following 2 times of washing with fresh target cell culture medium.

Tetracycline was added to the culture media to a concentration of 1 μg/ml and incubated for an additional 48 hours. Thereafter, the culture media were recovered and measured for their HeLa cell killing ability.

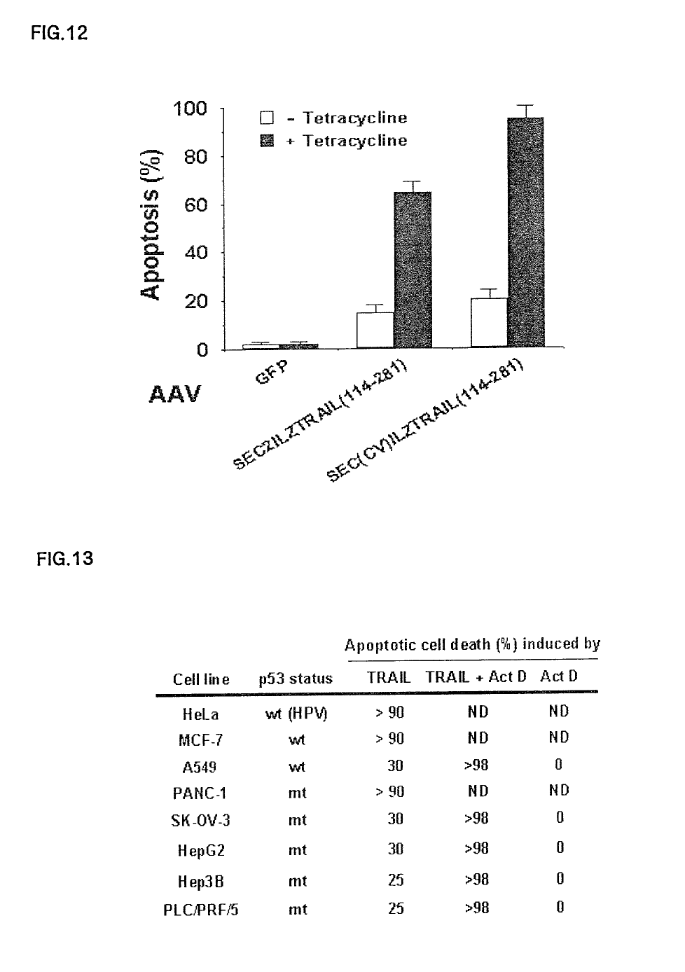

The culture medium of HeLa cells plated in 12 wells was replaced with the test culture medium (1 ml) and incubated for 12 hours. Cell viability was measured by a spectrophotometric analysis using crystal violet or by counting microscopically the dead cells with typical apoptotic morphology (FIG. 12).

As can be seen from FIG. 12, SEC2ILZTRAIL(114-281) or SEC(CV)ILZTRAIL(114-281) sequences packaged into virus particles in combination with the Tet-On system of the present invention [also] equally exhibits target cell killing activity. This result implies that SEC2ILZTRAIL(114-281) and SEC(CV)ILZTRAIL(114-281) can also be expressed by AAV particles in a Tetracycline-inducible manner and secreted into the culture medium, as was demonstrated in the case of the pAAVdwSEC2ILZTRAIL(114-281) and pAAVdwSEC(CV)ILZTRAIL(114-281) vectors.

Muscle cells represent a very good target tissue for AAV particles, as established in many studies. It has been well known that upon transduction of a target gene by AAV particles, muscle cells express the target gene almost permanently.

Hence, the above results suggest that, when the cassette of the present invention for recombinant TRAIL production is applied to the body using AAV particles, rTRAIL can be produced in muscle cells and secreted into the circulation almost permanently in a Tetracycline/Doxycycline-inducible manner.

### Example 7

Combination Therapy

In general, combination therapies produce a better prognosis than individual therapies in cancer treatment. Thus, effects of the combined therapy of rTRAIL protein and a chemotherapeutic agent ActD were tested.

To test the ActD-enhanced apoptotic activity of recombinant human TRAIL(114-281) protein, each cell line was plated in 12 wells and incubated with recombinant human TRAIL(114-281) protein (100 ng/ml) alone, recombinant human TRAIL(114-281) protein (100 ng/ml) plus ActD (100 ng/ml), or ActD (100 ng/ml) alone for 12 hours. Cell viability was measured by a spectrophotometric analysis using crystal violet. The results are shown in FIG. 13 “ND” stands for “not determined”.

As can be seen from FIG. 13, the apoptotic activity of recombinant human TRAIL(114-281) protein was significantly enhanced by ActD. With the aid of ActD, recombinant human TRAIL(114-281) protein killed most target cells tested regardless of their intrinsic resistance against the recombinant human TRAIL(114-281) protein alone.

Accordingly, recombinant human TRAIL(114-281) protein fortified by ActD is expected to be a therapeutic agent for a variety of cancers.

As described and demonstrated in detail, the pAAVdw vector and the pCMVdw vector and the TRAIL DNA cassette produced in the present invention have been shown to elicit a satisfactory apoptotic activity in a test using HeLa cells. The measurement of cell viability for the virus packaged from the pAAVdw recombinant expression vector indicated a typical apoptotic activity.

The gene therapy using AAV particles harboring a pAAVdw vector containing the DNA cassette of the present invention for the production of secretable trimeric rTRAIL allows the direct endogenous generation of a high quality, therapeutically useful rTRAIL protein. The present invention using AAV particles also allows the endogenous production of the rTRAIL almost permanently with only a single injection, owing to the nature of AAV. In addition, by using a Tet-On inducible system, the present invention allows for a high quality rTRAIL to be produced in the body of a patient almost permanently in a regulated manner with inexpensive Tetracycline or Doxycycline. Therefore, a cancer therapy utilizing the present invention is expected to show superiority over known anticancer drugs in terms of the cost burden on patients as well as in terms of facility.

In addition, the present invention can be utilized in the mass production of a recombinant trimeric TRAIL protein or in gene therapy.

What is claimed is:

1. A DNA cassette for the production of a secretable recombinant trimeric TRAIL protein, comprising a secretion signal (SS) sequence, a trimer-forming domain (TFD), and a TRAIL(114-281) coding cDNA.

2. The DNA cassette of claim 1, wherein the secretion signal sequence is SEC2 or SEC(CV).

3. The DNA cassette of claim 1, wherein the trimer-forming domain is ILZ.

4. A recombinant expression vector comprising the DNA cassette of any of claims 1 to 3.

5. A cell line transfected with the recombinant expression vector of claim 4.

6. A secretable recombinant trimeric TRAIL protein produced by the cell line of claim 5.

7. A mammalian expression vector pCMVdw, which can constitutively express an inserted foreign gene.

8. The pCMVdw vector of claim 7, which comprises the secretion signal sequence SEC2 or SEC(CV) such that the protein encoded by the inserted foreign gene can be secreted into the extracellular space.

9. The pCMVdw vector of claim 7, which comprises the trimer-forming domain ILZ such that the protein encoded by the inserted foreign gene can form a trimer.

10. The pCMVdw vector of claim 7, which comprises the secretion signal sequence SEC2 or SEC(CV) and the trimer-forming domain ILZ such that the protein encoded by the inserted foreign gene can be secreted into the extracellular space and form a trimer.

11. The recombinant expression vector of claim 4, wherein the expression vector is pCMVdw.

12. An adeno-associated virus (AAV) expression vector pAAVdw, which can express an inserted foreign gene in a Tetracycline/Doxycycline-inducible manner by a feed-forward amplification loop of a Tet-On system.

13. The recombinant expression vector of claim 4, wherein the expression vector is pAAVdw.

14. The pAAVdw vector of claim 12, which comprises the secretion signal sequence SEC2 or SEC(CV) such that the protein encoded by the inserted foreign gene can be secreted into the extracellular space.

15. The pAAVdw vector of claim 12, which comprises the trimer-forming domain ILZ such that the protein encoded by the inserted foreign gene can form a trimer.

16. The pAAVdw vector of claim 12, which comprises the secretion signal sequence SEC2 or SEC(CV) and the trimer-forming domain ILZ such that the protein encoded by the inserted foreign gene can be secreted into the extracellular space and form a trimer.

17. A virus particle packaged from the recombinant expression vector of claim 13.

18. A cell line transformed by the virus of claim 17.

19. A secretable recombinant trimeric TRAIL protein produced by the cell line of claim 18.

20. A pharmaceutical composition for the treatment of cancer and other diseases, comprising a therapeutically effective amount of the recombinant expression vector of claim 4 as the active ingredient.

21. A pharmaceutical composition for the treatment of cancer and other diseases, comprising a therapeutically effective amount of the recombinant expression vector of claim 11 as the active ingredient.

22. A pharmaceutical composition for the treatment of cancer and other diseases, comprising a therapeutically effective amount of the recombinant expression vector of claim 13 as the active ingredient.

23. A pharmaceutical composition for the treatment of cancer and other diseases, comprising a therapeutically effective amount of the virus of claim 17 as the active ingredient.

24. The pharmaceutical composition of claim 20, further comprising ActD.

25. The pharmaceutical composition of claim 23, further comprising ActD.
